(19) United States
(12) Patent Application Publication
(10) Pub. No.: US 2012/0109238 A1
(43) Pub. Date: May 3, 2012

(54) AUTOMATIC PERSONALIZATION OF PARAMETER SETTINGS AND ALGORITHMS IN A MEDICAL DEVICE

(75) Inventors: Amisha S. Patel, Maple Grove, MN (US); Luc R. Mongeon, Minneapolis, MN (US); Kenneth M. Riff, Wayzata, MN (US)

(73) Assignee: Medtronic, Inc., Minneapolis, MN (US)

(21) Appl. No.: 12/915,863

(22) Filed: Oct. 29, 2010

(51) Int. Cl. A61N 1/08; A61N 1/362; A61N 1/39

(52) U.S. Cl. 607/5; 607/60; 607/32

(57) ABSTRACT

A system includes a data retrieval module and a determination module. The data retrieval module receives a command from a user, the command indicating a first implantable medical device (IMD) and a second IMD. The data retrieval module also retrieves a first set of data from the first IMD in response to the command and retrieves a second set of data from a datastore. The second set of data includes data retrieved from the first IMD and stored in the datastore prior to receiving the command. The determination module determines a third set of data based on the first and second sets of data and transfers the third set of data to the second IMD.

### TECHNICAL FIELD

The disclosure relates to systems and methods for programming implantable medical devices, and, more particularly, to systems and methods for programming implantable medical devices based on longitudinal patient data.

### BACKGROUND

A variety of implantable medical devices are employed to provide therapy to patients and/or monitor physiological parameters of patients. For example, an implantable medical device (IMD), such as a cardiac pacemaker or a cardioverter-defibrillator, may provide therapeutic electrical stimulation to a patient via electrodes carried by one or more implantable leads. Such an IMD may provide the electrical stimulation based on sensed physiological parameters of the patient, such as electrical signals sensed via the electrodes carried by the implantable leads. The IMD may include a memory that stores various data related to delivery of therapy and/or monitoring of the patient. In a pacemaker or cardioverter-defibrillator, the stored data may include, for example, electrogram waveforms (EGMs), marker channel data, and programmable parameters, such as pacing parameters and tachyarrhythmia detection parameters.

An IMD may reach its end of life, e.g., when its power source is depleted, after being implanted in a patient for a period of time. For example, end of life for a cardioverter-defibrillator and cardiac pacemaker may be approximately 5-7 years and 10-12 years, respectively. A currently implanted IMD is typically replaced by another IMD (i.e., a replacement IMD) prior to end of life. The procedure involving replacement of the currently implanted IMD with the replacement IMD may be referred to as a “change-out procedure.”

During a change-out procedure, the implanted IMD may be interrogated, e.g., using a programmer, and the data retrieved from the implanted IMD may be stored or printed out. Subsequently, a clinician may use the programmer to input program settings into the replacement IMD based on the settings of the previously implanted IMD. In some examples, the program settings may be manually entered into the programmer by the clinician and then transferred to the replacement IMD. Additionally, during some change-out procedures the clinician may review the patient's history to determine which settings are appropriate for the replacement IMD prior to entering the program settings for the replacement IMD.

### SUMMARY

The transfer of data to a replacement IMD during a change-out procedure may be a manual procedure that is labor intensive and has a high potential for error. Clinician review of the patient's history to determine settings for the replacement IMD may also be a manual process that has a high potential for error.

In some examples, the disclosure is directed to techniques for determining replacement data for a replacement IMD during a change-out procedure. Such techniques according to the present disclosure include retrieving data from a currently implanted IMD, retrieving data from a datastore, and determining the replacement data to program into the replacement IMD based on the data from the currently implanted IMD and the data from the datastore. Data retrieved from the datastore may include longitudinal patient data. Longitudinal patient data may include any data that is related to the patient undergoing the change-out procedure. For example, longitudinal patient data may include data retrieved from the implanted IMD over the duration of time during which the IMD was implanted in the patient (e.g., over a period of years). Data retrieved from the datastore may also include data related to other patients (i.e., cross-patient data). For example, cross-patient data may include data retrieved from IMDs of other patients over the duration of time during which the IMDs were implanted in the other patients (e.g., over a period of years). Accordingly, the techniques of the present disclosure include determining replacement data for a replacement IMD based on at least one of IMD data retrieved from the patient undergoing the procedure, longitudinal patient data, or cross-patient data. In some examples, replacement data for replacement IMD may be based on knowledge of new and enhanced functionality of the replacement device.

Determining replacement IMD parameter settings based on longitudinal patient data provides various benefits. For example, determining replacement IMD parameter settings based on longitudinal patient data may provide for personalization of IMD parameters upon implant of the replacement IMD. The personalized IMD parameters may perform more effectively than nominal factory programmed parameters of the replacement IMD since the personalized IMD parameters are based on data indicating effective treatment of the patient in the past.

A clinician may also benefit from significant time savings using a system that determines replacement IMD parameter settings based on longitudinal patient data from a datastore. Instead of requiring manual review of the patient's history, the techniques of the present disclosure may automatically analyze a large amount of longitudinal patient data in the datastore and present replacement IMD parameters, based on the analysis, to the clinician for review. Accordingly, the clinician may avoid the manual process of reviewing the patient's history in order to determine replacement IMD parameters, thus reducing an amount of time and risk of error associated with manual review.

In one feature of the present disclosure, a system comprises a data retrieval module and a determination module. The data retrieval module receives a command from a user, the command indicating a first implantable medical device (IMD) and a second IMD, retrieves a first set of data from the first IMD in response to the command, and retrieves a second set of data from a datastore. The second set of data includes data retrieved from the first IMD and stored in the datastore prior to receiving the command. The determination module determines a third set of data based on the first and second sets of data transfers the third set of data to the second IMD.

In another feature of the present disclosure, a method comprises receiving a command from a user, the command indicating a first implantable medical device (IMD) and a second IMD, retrieving a first set of data from the first IMD in response to the command, and retrieving a second set of data from a datastore. The second set of data includes data retrieved from the first IMD and stored in the datastore prior to receiving the command. The method further comprises determining a third set of data based on the first and second sets of data and transferring the third set of data to the second IMD.

In another feature of the present disclosure, a system comprises means for receiving a command from a user, the command indicating a first implantable medical device (IMD) and a second IMD, means for retrieving a first set of data from the first IMD in response to the command, and means for retrieving a second set of data from a datastore. The second set of data includes data retrieved from the first IMD and stored in the datastore prior to receiving the command. The system further comprises means for determining a third set of data based on the first and second sets of data and means for transferring the third set of data to the second IMD.

In another feature of the present disclosure, a computer-readable storage medium comprises instructions that cause a programmable processor to receive a command from a user, the command indicating a first implantable medical device (IMD) and a second IMD, and to retrieve a first set of data from the first IMD in response to the command. The computer-readable storage medium further comprises instructions that cause the programmable processor to retrieve a second set of data from a datastore. The second set of data includes data retrieved from the first IMD and stored in the datastore prior to receiving the command. Additionally, the computer-readable storage medium further comprises instructions that cause the programmable processor to determine a third set of data based on the first and second sets of data and transfer the third set of data to the second IMD.

In still other features of the present disclosure, a system comprises a data retrieval module and a determination module. The data retrieval module receives an update request, retrieves a first set of data from an IMD implanted in a patient in response to the update request, and retrieves a second set of data from a datastore in response to the update request. The second set of data includes data retrieved from the IMD and stored in the datastore prior to receiving the update request. The determination module determines a third set of data based on the first and second sets of data and transfers the third set of data to the IMD.

In another feature of the present disclosure, a method comprises receiving an update request, retrieving a first set of data from an IMD implanted in a patient in response to the update request, and retrieving a second set of data from a datastore in response to the update request. The second set of data includes data retrieved from the IMD and stored in the datastore prior to receiving the update request. The method further comprises determining a third set of data based on the first and second sets of data, and transferring the third set of data to the IMD.

In another feature of the present disclosure, a system comprises means for receiving an update request, means for retrieving a first set of data from an IMD implanted in a patient in response to the update request, and means for retrieving a second set of data from a datastore in response to the update request. The second set of data includes data retrieved from the IMD and stored in the datastore prior to receiving the update request. The system further comprises means for determining a third set of data based on the first and second sets of data, and means for transferring the third set of data to the IMD.

In another feature of the present disclosure, a computer-readable storage medium comprises instructions that cause a programmable processor to receive an update request and retrieve a first set of data from an IMD implanted in a patient in response to the update request. The computer-readable storage medium further comprises instructions that cause the programmable processor to retrieve a second set of data from a datastore in response to the update request. The second set of data includes data retrieved from the IMD and stored in the datastore prior to receiving the update request. Additionally, the computer-readable storage medium further comprises instructions that cause the programmable processor to determine a third set of data based on the first and second sets of data, and transfer the third set of data to the IMD.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram of an example system comprising an implantable medical device (IMD) for delivering stimulation therapy to a heart of a patient via implantable leads.

FIG. 2 is a conceptual diagram of the IMD and the implantable leads of the system of FIG. 1 in conjunction with the heart.

FIG. 3 is a conceptual diagram of the IMD of FIG. 1 coupled to a different configuration of leads.

FIG. 4 is a functional block diagram illustrating an example configuration of the IMD of FIG. 1.

FIG. 5 is a functional block diagram of an example programmer.

FIG. 6 is a functional block diagram illustrating retrieval of data from a first IMD that is being removed from a patient and transmission of data to a replacement IMD that is being implanted in the patient.

FIG. 7 is a functional block diagram of the example programmer communicating with a remote device that determines replacement IMD data during a change-out procedure.

FIG. 8 is a functional block diagram of an example implementation of the remote device of FIG. 7.

FIG. 9 illustrates an example method for determining replacement IMD data during a change-out procedure.

FIG. 10 is a functional block diagram illustrating retrieval of data from an IMD and transmission of updated data to the IMD.

FIG. 11 is another functional block diagram illustrating retrieval of data from the IMD and transmission of updated data to the IMD.

FIG. 12 is a functional block diagram of an example implementation of the remote device that retrieves data from the IMD and transmits updated data to the IMD.

FIG. 13 illustrates an example method for determining updated IMD data during an update procedure.

### DETAILED DESCRIPTION

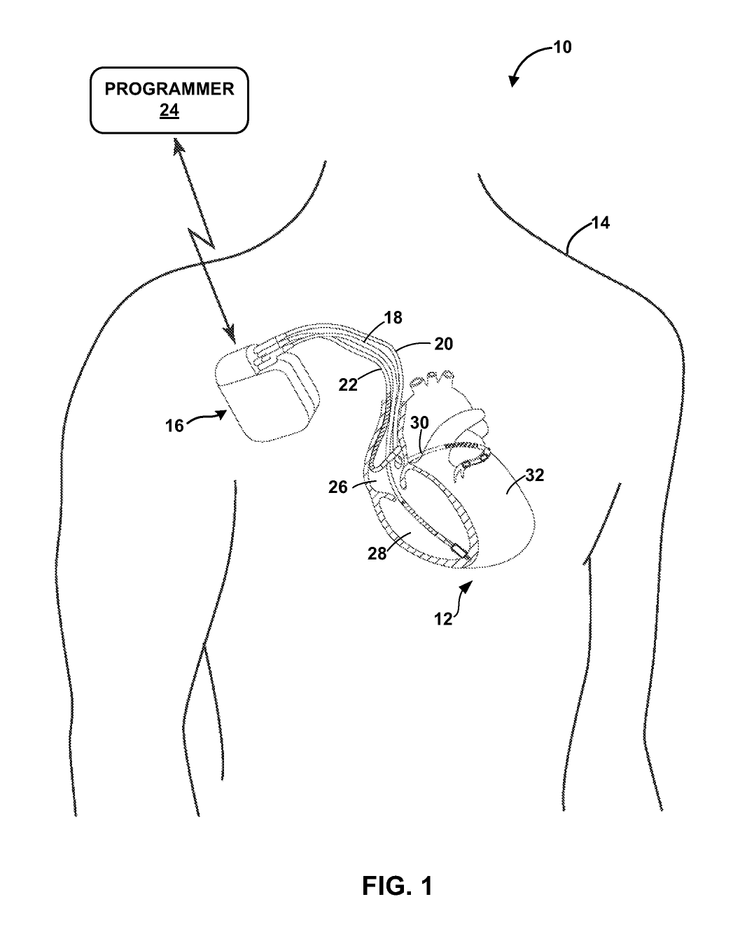

FIG. 1 is a conceptual diagram of an example system 10 that may be used to diagnose conditions of and provide therapy to a heart 12 of a patient 14. System 10 includes an IMD 16, which is coupled to leads 18, 20, and 22. For example, IMD 16 may be an implantable pacemaker, cardioverter, and/or defibrillator that provides electrical signals to heart 12 using one or more of leads 18, 20, 22.

Leads 18, 20, 22 extend into heart 12 of patient 14. Leads 18, 20, 22 sense electrical activity of heart 12 and/or deliver electrical stimulation to heart 12. Right ventricular (RV) lead 18 extends into right ventricle 28 of heart 12 through one or more veins (not shown), the superior vena cava (not shown), and right atrium 26. Left ventricular (LV) coronary sinus lead 20 extends through one or more veins, the vena cava, right atrium 26, and into coronary sinus 30 to a region adjacent to the free wall of left ventricle 32 of heart 12. Right atrial (RA) lead 22 extends into right atrium 26 of heart 12 through one or more veins and the vena cava.

System 10 includes a programmer 24 that communicates with IMD 16. Programmer 24 may be a handheld computing device, desktop computing device, a networked computing device, etc. Accordingly, programmer 24 may be a computing device that includes a computer-readable storage medium having instructions that cause a processor of programmer 24 to provide the functions attributed to programmer 24 in the present disclosure. System 10 may also include a patient monitor 25. Patient monitor 25 may be a device that reads data from IMD 16 and uploads the data to a server, e.g., automatically or in response to a command from a patient or other user.

Although shown together in FIG. 1 for ease of illustration, programmer 24 and patient monitor 25 may, but typically will not, be co-located. Instead, programmer 24 and patient monitor 25 may individually communicate with IMD 16 when co-located with IMD 16 at respective times. For example, programmer 24 may be used by a clinician in a clinical setting to communicate with IMD 16 (e.g., during a follow-up), and patient monitor 25 may communicate with IMD 16 in a patient's home, automatically or in response to a user command.

Programmer 24 may retrieve data stored in IMD 16 and/or program the operation of IMD 16, e.g., to monitor patient 14 and/or to provide various therapies to patient 14. Accordingly, a user may retrieve data from IMD 16 and program IMD 16 using programmer 24. For example, the user may include a physician, a technician, a surgeon, an electrophysiologist, or other clinician.

Data retrieved by programmer 24 from IMD 16, and data transmitted from programmer 24 to IMD 16, e.g., during programming of IMD 16, may include any of the various types of data stored in memory of IMD 16. For example only, data stored in IMD 16 may include waveforms measured by IMD 16, marker channel data associated with the waveforms, programmable parameters of IMD 16, and algorithms implemented by IMD 16. The various types of data that may be transferred to, retrieved from, and stored in IMD 16 are discussed in further detail hereinafter.

Data retrieved from IMD 16 using programmer 24 includes waveforms that indicate electrical activity of heart 12. The waveforms retrieved from the IMD 16 may be referred to as cardiac electrogram waveforms. Cardiac electrogram waveforms stored by IMD 16 and retrieved by programmer 24 may be referred to as “EGMs.” Data retrieved from IMD 16 using programmer 24 may also include marker channel data. Marker channel data may indicate the occurrence and timing of sensing, diagnosis, and therapy events associated with IMD 16.

Programmer 24 may retrieve various types of data from IMD 16. For example, programmer 24 may retrieve EGMs from IMD 16, trends in the rhythm of heart 12 over time, or other sensed physiological parameters of the patient 14, such as intracardiac or intravascular pressure, activity, posture, respiration, or thoracic impedance. Additionally, data retrieved from IMD 16 may include information regarding the performance or integrity of IMD 16 or other components of system 10, such as leads 18, 20, 22, or a power source of IMD 16.

A user may program IMD 16 using programmer 24. Programming IMD 16 may include, for example, setting values for operational parameters (e.g., pulse rate, width and amplitude), programming a therapy progression, selecting electrodes used to deliver defibrillation pulses, selecting waveforms for the defibrillation pulses, or selecting or configuring a fibrillation detection algorithm for the IMD 16.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near an implant site of IMD 16 in order to improve the quality or security of communication between IMD 16 and programmer 24.

Patient monitor 25 may be a handheld computing device, desktop computing device, a networked computing device, etc. Patient monitor 25 may include similar functionality as programmer 24. Specifically, patient monitor 25 may retrieve various types of stored or real-time data from IMD 16. For example, patient monitor 25 may retrieve EGMs from IMD 16, trends in the rhythm of heart 12 over time, or other sensed physiological parameters of patient 14, such as intracardiac or intravascular pressure, activity, posture, respiration, or thoracic impedance. Patient monitor 25 may also retrieve marker channel data from IMD 16. Additionally, patient monitor 25 may retrieve information regarding the performance or integrity of IMD 16 or other components of diagnostic system 10, such as leads 18, 20, 22, or a power source of IMD 16. Patient monitor 25 may transfer data from IMD 16 to a clinic or to a remote device through a network.

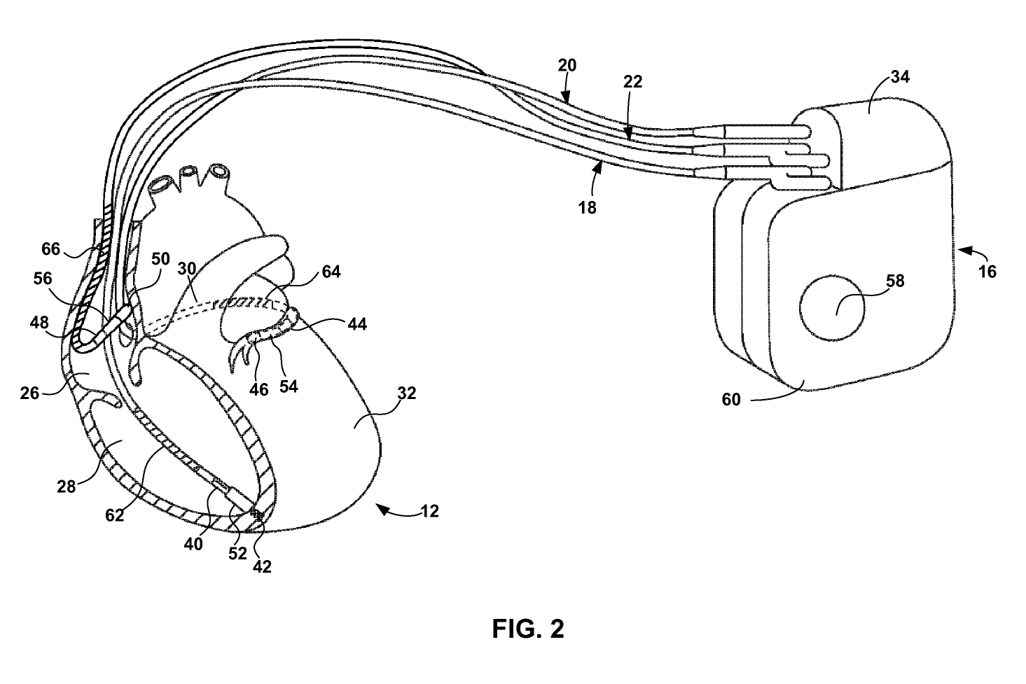

FIG. 2 is a conceptual diagram illustrating IMD 16 and leads 18, 20, 22 of system 10 in greater detail. IMD 16 includes a housing 60 and a connector block 34. Leads 18, 20, 22 are mechanically and electrically coupled to IMD 16 via connector block 34. Housing 60 may enclose a signal generator that generates therapeutic stimulation, such as cardiac pacing pulses and cardioversion or defibrillation therapy, as well as a sensing module for monitoring the rhythm of heart 12. Leads 18, 20, 22 are coupled to a signal generator and a sensing module of IMD 16 via connector block 34. IMD 16 may sense electrical signals attendant to the depolarization and repolarization of heart 12 via leads 18, 20, 22. IMD 16 may also provide electrical stimulation to heart 12 via leads 18, 20, 22.

IMD 16 may provide pacing pulses to heart 12 based on the electrical signals sensed within heart 12. IMD 16 may also provide defibrillation and/or cardioversion therapy to heart 12. For example, IMD 16 may detect arrhythmia of heart 12, such as tachycardia or fibrillation of the ventricles 28 and 32, and deliver cardioversion or defibrillation therapy to heart 12, e.g., in the form of electrical pulses. In some implementations, IMD 16 may be programmed to deliver a progression of therapies, e.g., pulses with increasing energy levels, until a tachyarrhythmia of heart 12 is stopped. IMD 16 may detect tachycardia or fibrillation employing one or more tachycardia or fibrillation detection techniques known in the art.

Leads 18, 20, 22 include electrodes 40, 42, 44, 46, 48, 50, 62, 64, and 66, respectively. IMD 16 may sense electrical signals via electrodes 40, 42, 44, 46, 48, 50, 62, 64, and 66. IMD 16 may also provide electrical stimulation to heart 12 using electrodes 40, 42, 44, 46, 48, 50, 62, 64, and 66. Although each of leads 18, 20, 22 of FIG. 2 includes three electrodes, other configurations of electrodes are contemplated. For example, each of the three leads 18, 20, 22 may include more or less than three electrodes.

Bipolar electrodes 40 and 42 are located adjacent to the distal end of lead 18 in right ventricle 28. Bipolar electrodes 44 and 46 are located adjacent to the distal end of lead 20 in coronary sinus 30. Bipolar electrodes 48 and 50 are located adjacent to the distal end of lead 22 in right atrium 26. There are no electrodes located in the left atrium in the illustrated example, however, other examples may include electrodes in left atrium.

Electrodes 40, 44, and 48 may take the form of ring electrodes. Electrodes 42, 46, and 50 may take the form of extendable helix tip electrodes mounted retractably within insulative electrode heads 52, 54, and 56, respectively. In other embodiments, one or more of electrodes 42, 46, and 50 may take the form of small circular electrodes at the tip of a tined lead or other fixation element. Leads 18, 20, 22 also include elongated electrodes 62, 64, 66, respectively, which may take the form of a coil.

IMD 16 includes a housing electrode 58, which may be formed integrally with an outer surface of the hermetically-sealed housing 60 of IMD 16 or otherwise coupled to housing 60. Although a single housing electrode 58 is illustrated in FIG. 2, IMD 16 may include more or less than a single housing electrode 58.

IMD 16 may sense electrical signals attendant to the depolarization and repolarization of heart 12 via electrodes 40, 42, 44, 46, 48, 50, 58, 62, 64, and 66. The electrical signals are conducted to IMD 16 from the electrodes via the respective leads 18, 20, 22 or, in the case of housing electrode 58, a conductor coupled to housing electrode 58. IMD 16 may sense such electrical signals via any bipolar combination of electrodes 40, 42, 44, 46, 48, 50, 58, 62, 64, and 66. Furthermore, any of electrodes 40, 42, 44, 46, 48, 50, 62, 64, and 66 may be used for unipolar sensing in combination with housing electrode 58.

IMD 16 may deliver pacing pulses via a unipolar or bipolar combination of electrodes. IMD 16 delivers pacing pulses via bipolar combinations of electrodes 40, 42, 44, 46, 48 and 50 to produce depolarization of cardiac tissue of heart 12. IMD 16 may deliver pacing pulses via any of electrodes 40, 42, 44, 46, 48 and 50 in combination with housing electrode 58 in a unipolar configuration.

IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12.

The electrode configuration of system 10 illustrated in FIGS. 1 and 2 is merely one example electrode configuration. In other examples, a system may include epicardial leads and/or patch electrodes instead of or in addition to the transvenous leads 18, 20, 22 illustrated in FIGS. 1-2.

Although IMD 16 of FIGS. 1-2 is coupled to three leads 18, 20, 22, other lead configurations are contemplated. In other words, the number of leads coupled to IMD 16 and the locations of the leads relative to heart 12 may vary. For example, in some alternative implementations, system 10 may include an additional lead or lead segment (not shown in FIGS. 1-2) that deploys one or more electrodes within the left atrium, vena cava, or other vein. The additional lead may allow for alternative electrical sensing configurations that may provide improved sensing accuracy in some patients.

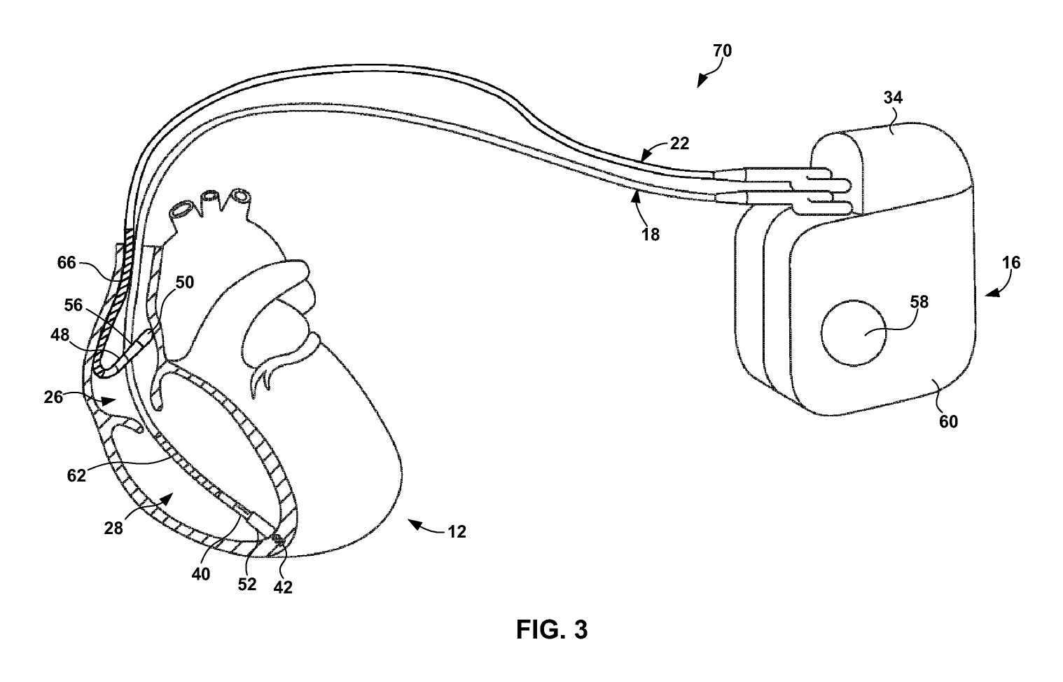

FIG. 3 is a conceptual diagram illustrating another lead configuration. A system 70, similar to system 10, includes two leads 18, 22, rather than three leads. Leads 18, 22 are implanted within right ventricle 28 and right atrium 26, respectively. System 70 shown in FIG. 3 may be useful for providing defibrillation and pacing pulses to heart 12. The systems and methods of the present disclosure may also be implemented in system 70.

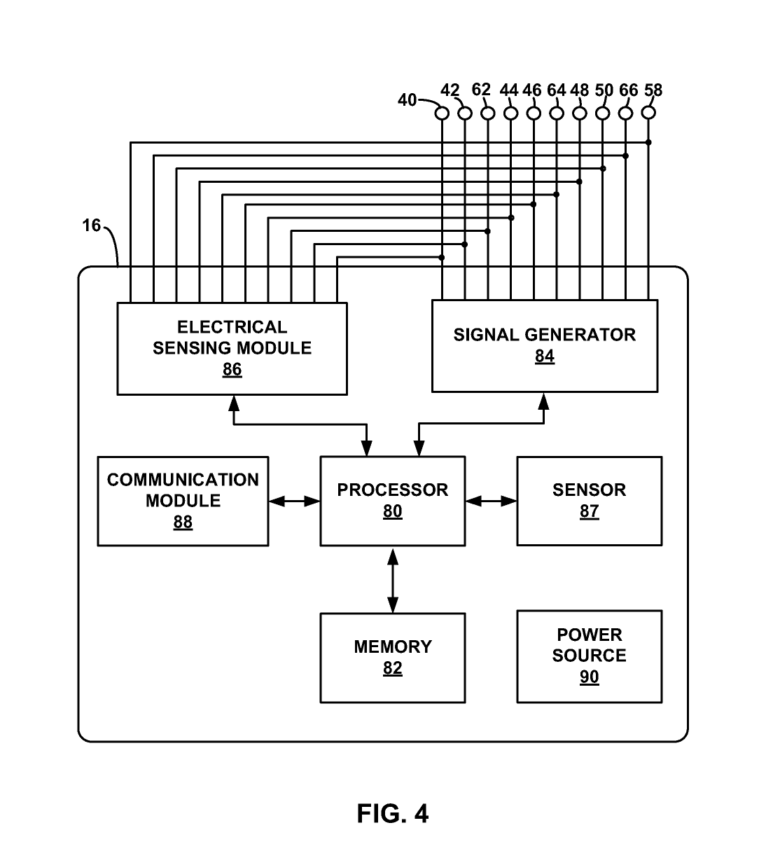

FIG. 4 is a functional block diagram illustrating an example configuration of IMD 16. IMD 16 includes a processor 80, memory 82, a signal generator 84, an electrical sensing module 86, a sensor 87, a communication module 88, and a power source 90. Memory 82 may include computer-readable instructions that, when executed by processor 80, cause IMD 16 and processor 80 to perform various functions attributed to IMD 16 and processor 80 herein. Memory 82 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other digital media.

Processor 80 may include any one or more of a microprocessor, a microcontroller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more microcontrollers, one or more DSPs, one or more ASICs, or one or more FPGAs, as well as other discrete or integrated logic circuitry. The functions attributed to processor 80 herein may be embodied as software, firmware, hardware or any combination thereof.

Signal generator 84 is electrically coupled to electrodes 40, 42, 44, 46, 48, 50, 58, 62, 64, and 66, e.g., via conductors of the respective leads 18, 20, 22, or, in the case of housing electrode 58, via an electrical conductor disposed within housing 60 of IMD 16. Signal generator 84 is configured to generate and deliver electrical stimulation therapy to heart 12. For example, signal generator 84 may deliver defibrillation pulses to heart 12 via at least two electrodes 58, 62, 64, 66. Signal generator 84 may deliver pacing pulses via the ring electrodes 40, 44, 48 coupled to leads 18, 20, and 22, respectively, and/or the helical electrodes 42, 46, and 50 of leads 18, 20, and 22, respectively. In some implementations, signal generator 84 delivers pacing, cardioversion, or defibrillation stimulation in the form of electrical pulses. In other implementations, signal generator 84 may deliver one or more of these types of stimulation in the form of other signals, such as sine waves, square waves, or other substantially continuous time signals.

Processor 80 controls signal generator 84 to deliver stimulation therapy to heart 12. Processor 80 may control signal generator 84 to deliver stimulation according to a selected one or more therapy programs, which may be stored in memory 82. For example, processor 80 may control signal generator 84 to deliver electrical pulses with amplitudes, pulse widths, frequencies, or electrode polarities specified by the selected one or more therapy programs.

Processor 80 may select which of electrodes 40, 42, 44, 46, 48, 50, 58, 62, 64, and 66 delivers electrical pulses. For example, signal generator 84 may include a switch module that processor 80 may use to select, e.g., via a data/address bus, which of the available electrodes are used to deliver pacing, cardioversion, or defibrillation pulses. The switch module may include a switch array, switch matrix, multiplexer, or any other type of switching device suitable to selectively couple electrical pulses to electrodes selected by processor 80.

Electrical sensing module 86 monitors signals from at least one of electrodes 40, 42, 44, 46, 48, 50, 58, 62, 64 or 66 in order to monitor electrical activity of heart 12. Processor 80 may select which of electrodes 40, 42, 44, 46, 48, 50, 58, 62, 64, and 66 function as sense electrodes. For example, electrical sensing module 86 may include a switch module that processor 80 may use to select, e.g., via a data/address bus, which of the electrodes are used to monitor electrical activity of heart 12.

Electrical sensing module 86 may include multiple detection channels, each of which may comprise an amplifier. In response to the signals from processor 80, the switch module within the electrical sensing module 86 may couple selected electrodes to each of the detection channels.

Processor 80 may include a timing and control module, which may be embodied as hardware, firmware, software, or any combination thereof. The timing and control module may comprise a dedicated hardware circuit, such as an ASIC, separate from other processor 80 components, such as a microprocessor, or a software module executed by a component of processor 80, which may be a microprocessor or ASIC. The timing and control module may implement programmable counters. If IMD 16 is configured to generate and deliver pacing pulses to heart 12, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control module within processor 80 may include atrial and ventricular pacing escape intervals, refractory periods during which sensed P-waves and R-waves are ineffective to restart timing of the escape intervals, and the pulse widths of the pacing pulses. As another example, the timing and control module may withhold sensing from one or more channels of sensing module 86 for a time interval during and after delivery of electrical stimulation to heart 12. The durations of these intervals may be determined by processor 80 in response to stored data in memory 82. The timing and control module of processor 80 may also determine the amplitude of the cardiac pacing pulses.

A portion of memory 82 may be configured as a plurality of recirculating buffers, capable of holding a series of measured intervals, which may be analyzed by processor 80 to determine whether the patient's heart 12 is presently exhibiting atrial or ventricular tachyarrhythmia. Processor 80 may detect tachyarrhythmia using any suitable tachyarrhythmia detection algorithm. In the event that processor 80 detects an atrial or ventricular tachyarrhythmia, an anti-tachyarrhythmia pacing regimen may be loaded by processor 80 and implemented using signal generator 84.

Signal generator 84 may include a high voltage charge circuit and a high voltage output circuit when IMD 16 is configured to generate and deliver defibrillation pulses to heart 12. In response to the detection of atrial or ventricular fibrillation or tachyarrhythmia requiring a cardioversion pulse, processor 80 may activate a cardioversion/defibrillation therapy using the high voltage charge circuit and the high voltage output circuit. Following delivery of the fibrillation or tachycardia therapy, processor 80 may return signal generator 84 to a cardiac pacing function and await the next successive interrupt due to pacing or the occurrence of a sensed atrial or ventricular depolarization.

IMD 16 may include one or more sensors, such as sensor 87. Sensor 87 may comprise a pressure sensor (e.g., a capacitive sensor) that senses intracardiac or other cardiovascular pressure. Sensor 87 may comprise a motion sensor. The motion sensor may be, for example, an accelerometer or piezoelectric element. Sensor 87 may also comprise a heart sound sensor, or any sensor capable of generating a signal that varies as a function of mechanical activity, e.g., contraction of heart 12. Processor 80 may receive one or more signals from sensor 87 or a plurality of sensors. Processor 80 may monitor, among other things, the mechanical activity of heart 12 based on signals from the one or more sensors.

Sensor 87 may be positioned in various locations in diagnostic system 10. For example, sensor 87 may be located within housing 60, outside of housing 60, or on or within on or more of leads 18, 20, 22. Sensor 87 may communicate with IMD 16 via wireless communication when sensor 87 is located outside of housing 60. In some implementations, sensor 87 may be external (i.e., not implanted).

Communication module 88 includes any suitable hardware, firmware, software or any combination thereof for communicating with another device, such as programmer 24. Under the control of processor 80, communication module 88 may receive downlink telemetry from and send uplink telemetry to programmer 24 with the aid of an antenna (not shown), which may be internal and/or external. Processor 80 may provide the data to be uplinked to programmer 24 and the control signals for telemetry circuitry within communication module 88, e.g., via an address/data bus.

Processor 80 may transmit atrial and ventricular heart signals (e.g., EGMs) detected by atrial and ventricular sense amplifier circuits within electrical sensing module 86 to programmer 24. Additionally, programmer 24 may interrogate IMD 16 to receive the EGMs. Processor 80 may provide stored and/or real-time EGMs to programmer 24 via communication module 88 in response to the interrogation.

Processor 80 may store the EGMs in memory 82, and retrieve the stored EGMs from memory 82. Processor 80 may also generate marker channel data and store marker channel data in memory 82. Marker channel data may indicate the occurrence and timing of sensing, diagnosis, and therapy events, e.g., P-waves, R-waves, tachyarrhythmia (e.g., tachycardia or fibrillation), pacing pulses, anti-tachycardia pacing (ATP), cardioversion pulses, or defibrillation pulses, detected, diagnosed, or undertaken by IMD 16. Programmer 24 may interrogate IMD 16, via communication module 88, to receive the marker channel data. Processor 80 may also provide the marker channel data to programmer 24 in real-time via communication module 88, e.g., when the marker channel data is generated.

Processor 80 may store EGMs corresponding to physiological episodes, such as tachyarrhythmias, in memory 82. For example, processor 80 may store EGMs for atrial and ventricular tachycardia and fibrillation episodes, in response to the detection of the tachycardia or fibrillation. Processor 80 may also store EGMs corresponding to nonsustained tachycardia (NST) in memory 82 in response to detection of the NST using any suitable NST detection technique. Programmer 24 may interrogate IMD 16, via communication module 88, to receive the stored EGMs.

Processor 80 may also store parametric data in memory 82. Parametric data may include, for example, impedance measurements, trends of impedance measurements, or statistical or other processed values determined based on impedance measurements. Other parametric data may include data indicating the current status of power source 90 of IMD 16. Programmer 24 may interrogate IMD 16, via communication module 88, to receive the parametric data. Processor 80 may also provide the parametric data to programmer 24 in real-time via the communication module 88, e.g., when the parametric data is measured.

The various components of IMD 16 are coupled to power source 90, which may include a rechargeable or non-rechargeable battery. A non-rechargeable battery may be capable of holding a charge for several years, while a rechargeable battery may be inductively charged from an external device, e.g., on a daily or weekly basis.

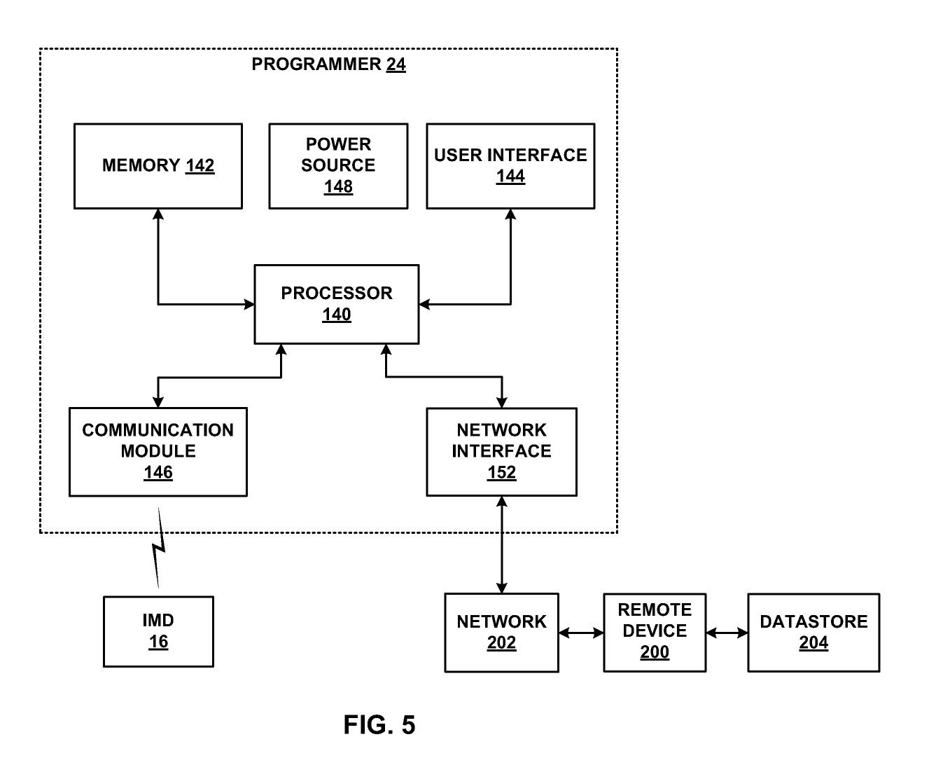

FIG. 5 is an example functional block diagram of programmer 24. Programmer 24 includes a processor 140, memory 142, a user interface 144, a communication module 146, a power source 148, and a network interface 152. Programmer 24 may be a dedicated hardware device with dedicated software for communicating with IMD 16. For example, programmer 24 may be a dedicated hardware device that programs operational parameters of IMD 16 and/or receives data from IMD 16. Alternatively, programmer 24 may be an off-the-shelf computing device, such as a desktop or laptop computer, running an application that enables programmer 24 to communicate with IMD 16 (i.e., receive data from IMD 16 and/or program IMD 16). Accordingly, programmer 24 represents any computing device capable of performing the functions attributed to programmer 24 in the present disclosure.

The user interacts with programmer 24 using user interface 144. User interface 144 may include an input device and a display (e.g., an LCD display). The user enters data into programmer 24 using the input device. The input device may include various devices for entering data. The input device may include a keypad, for example, an alphanumeric keypad or a reduced set of keys associated with particular functions of programmer 24. The input device may also include a freehand peripheral input device such as a mouse, a stylus, and a touchscreen.

Network interface 152 may communicate with a remote device 200 via a network 202. Accordingly, programmer 24 may communicate with remote device 200 via network interface 152. Remote device 200 may include, for example, a general purpose computing device such as an off-the-shelf desktop/laptop computer or server computer configured to communicate with programmer 24 via network 202. Network 202 may include various types of networks, such as a wide area network (WAN) and/or the Internet, for example. Although network 202 may represent a long range network (e.g., Internet or WAN), in some implementations, network 202 may be a shorter range network, such as a local area network (LAN).

Processor 140 can take the form of one or more microprocessors, DSPs, ASICs, FPGAs, programmable logic circuitry, or the like, and the functions attributed to processor 140 herein may be embodied as hardware, firmware, software or any combination thereof. Processor 140 of programmer 24 may provide any of the functionality ascribed herein, or otherwise perform any of the methods described herein.

Memory 142 may store instructions that cause processor 140 to provide the functionality ascribed to programmer 24 herein. Memory 142 may also store information used by processor 140 to provide the functionality ascribed to programmer 24 herein. Memory 142 may include any fixed or removable magnetic, optical, or electrical media, such as random access memory (RAM), read-only memory (ROM), compact-disc ROM (CD-ROM), hard or floppy magnetic disks, electrically erasable programmable ROM (EEPROM), or the like. Memory 142 may also store information that controls therapy delivery by IMD 16.

Processor 140 may communicate with remote device 200, which in turn communicates with datastore 204. Accordingly, programmer 24 may communicate with datastore 204. In other words, programmer 24 may transfer/retrieve data to/from datastore 204. In some examples, remote device 200 may be a server that communicates with programmer 24 to store data from programmer 24 in datastore 204 and retrieve data from datastore 204 for use by programmer 24. Datastore 204 may include any type of computer data storage or computer memory for storing data received from remote device 200. For example, datastore 204 may include magnetic storage media (e.g., hard disk drives), optical media (e.g., digital versatile disc drives), and/or solid state memory (e.g., dynamic random access memory or EEPROM).

As described above, patient monitor 25 may include similar functionality as programmer 24. Accordingly, patient monitor 25 may communicate with datastore 204. In other words, patient monitor 25 may transfer/retrieve data to/from datastore 204.

Datastore 204 may store data retrieved from IMD 16 over the period of time during which IMD 16 is implanted in patient 14. In other words, datastore 204 may store data retrieved from IMD 16 from the time of implant of IMD 16 until change-out of IMD 16 with a new IMD at the end of life of IMD 16.

In some implementations, remote device 200 and datastore 204 may include network technology and functionality similar to that provided by the Medtronic CareLink® Network developed by Medtronic, Inc., of Minneapolis, Minn. The data stored in datastore 204 may include, for example, EGMs, marker channel data, parametric data, or other sensed physiological parameters of patient 14, such as intracardiac or intravascular pressure, activity, posture, respiration, or thoracic impedance. In other implementations, remote device 200 and datastore 204 may represent or interface with a system configured to store electronic medical records (EMR), which may additionally or alternatively include other waveforms or medical information for patient 14.

Programmer 24 may communicate wirelessly with IMD 16, such as using RF communication or proximal inductive interaction. This wireless communication is possible through the use of communication module 146, which may be coupled to an internal antenna or an external antenna. For example, an external antenna may be included in a programming head (not shown) that is coupled to programmer 24. The programming head may be placed over heart 12 (i.e., IMD 16), as described above with reference to FIG. 1 to communicate with IMD 16. Communication module 146 may include similar functionality as communication module 88 of IMD 16.

Communication module 146 may also be configured to communicate with another computing device via wireless communication techniques. Examples of local wireless communication techniques may include RF communication according to the Institute of Electrical and Electronics Engineers (IEEE) 802.11, Bluetooth specification sets, infrared communication, e.g., according to the IrDA standard, or other standard or proprietary telemetry protocols.

Power source 148 delivers operating power to the components of programmer 24. Power source 148 may include a battery and/or adapter for connection to an alternating current (AC) wall socket.

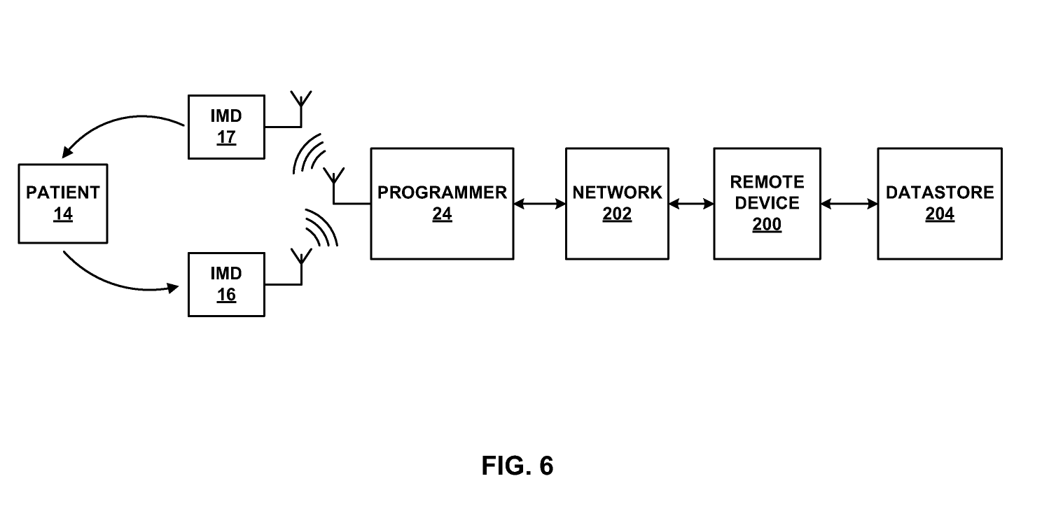

FIG. 6 illustrates transfer of data during a change-out procedure. In FIG. 6, IMD 16 (hereinafter “first IMD 16”) is removed from patient 14 during the change-out procedure. Subsequently, a replacement IMD 17 is implanted in patient 14 during the change-out procedure. FIG. 6 illustrates transmission of data from first IMD 16 to programmer 24 during the change-out procedure. FIG. 6 also illustrates reception of data by replacement IMD 17 during the change-out procedure.

Although transmission of data from first IMD 16 to programmer 24 may occur during the change-out procedure, in other examples, transmission of data from first IMD 16 may occur at a time prior to the change-out procedure. For example, data from first IMD 16 may be transmitted to datastore 204 prior to the change-out procedure and then subsequently used during the change-out procedure, e.g., retrieved from datastore 204 in response to a command entered during the change-out procedure.

Data retrieved from first IMD 16 during or before the change-out procedure may be referred to as “first IMD data.” Data transmitted to replacement IMD 17 during the change-out procedure may be referred to as “replacement IMD data.” In some examples, the first IMD data and the replacement IMD data may be the same. In other words, programmer 24 may copy the first IMD data from first IMD 16 and then transfer the first IMD data (i.e., the replacement IMD data) to replacement IMD 17. The first IMD data and the replacement IMD data may be the same when first IMD 16 and replacement IMD 17 are similar, or equivalent, devices.

In other examples, the replacement IMD data may be determined based on the first IMD data, and in some cases also based on data retrieved from datastore 204. For example, programmer 24 and/or remote device 200 may determine replacement IMD data that is different than first IMD data based on the first IMD data. Alternatively, programmer 24 and/or remote device 200 may determine replacement IMD data based on the first IMD data and the data retrieved from datastore 204. For example, programmer 24 and/or remote device 200 may determine replacement IMD data based on the first IMD data and data retrieved from datastore 204 associated with patient 14 and/or other patients.

Datastore 204 may include data related to patient 14. For example, data related to patient 14 may include data retrieved from first IMD 16 over the duration of time during which first IMD 16 was implanted in patient 14 (e.g., over a period of years). Data stored in datastore 204, relating to patient 14, may include data retrieved from first IMD 16 while first IMD 16 was implanted, such as stored EGMs and other waveforms. Datastore 204 may also include stored parametric data, such as impedance measurements, trends of impedance measurements, or statistical or other processed values determined based on impedance measurements. Datastore 204 may also include trends in the rhythm of heart 12 over time, or other sensed physiological parameters of the patient 14, such as intracardiac or intravascular pressure, activity, posture, or respiration. Datastore 204 may also include historic arrhythmia data, e.g., data regarding the occurrences, such as times of day of occurrences, of arrhythmias. Furthermore, datastore 204 may include typical P/R amplitudes of patient 14, typical percent pacing, typical capture thresholds, typical atrial fibrillation (AF) or atrial tachycardia (AT) burden, and typical episode frequency. Additionally, datastore 204 may also store values for programmable parameters of IMD 16 over time, such as alert thresholds, detection intervals, number of intervals detected (NID), and supra-ventricular tachycardia (SVT) and/or ventricular tachycardia (VT) templates. The data stored in datastore 204 that is related to patient 14 (i.e., the patient undergoing the change-out procedure) may be referred to hereinafter as “longitudinal patient data.” Longitudinal patient data is illustrated in the figures as “longitudinal patient data 210.”

In some examples, longitudinal patient data may also include data related to patient 14 that may have been acquired from sources other than IMD 16. For example, longitudinal patient data may include data acquired from EMR datastores. Data acquired from EMR datastores may include, but is not limited to, data related to other therapies used by patient 14 and efficacy of such therapies.

Datastore 204 may also include data related to other patients. Data related to other patients may include similar types of data as that stored in datastore 204 for patient 14. For example, datastore 204 may include data retrieved from IMDs of other patients over the duration of time during which the IMDs were implanted in the other patients (e.g., over a period of years). Data stored in datastore 204 that is related to patients other than patient 14 may be referred to hereinafter as “cross-patient data.” Cross-patient data is illustrated in the figures as “cross patient data 212.”

In some examples, cross-patient data may also include data relating to other patients that may have been acquired from sources other than IMDs implanted in the patients. For example, cross-patient data may include data compiled from EMR datastores that relates to other patients, e.g., other patients having similar conditions and/or medical histories as patient 14. Additionally, in some examples, cross-patient data may be derived from sources such as professional society guidelines, industry guidelines, results of medical studies, etc.

Based on data retrieved from datastore 204 and the first IMD data retrieved from first IMD 16, remote device 200 may determine the replacement IMD data for replacement IMD 17. Remote device 200 may then send the replacement IMD data to programmer 24 for transmission to replacement IMD 17 during the change-out procedure. Accordingly, during the change-out procedure, replacement IMD 17 may be programmed with replacement IMD data that is based on at least one of the first IMD data, the longitudinal patient data, and/or the cross-patient data. Although the present disclosure describes remote device 200 as determining the replacement IMD data based on the first IMD data, the longitudinal data, or the cross-patient data, in other implementations, programmer 24 and/or remote device 200, alone or in combination, may determine the replacement IMD data.

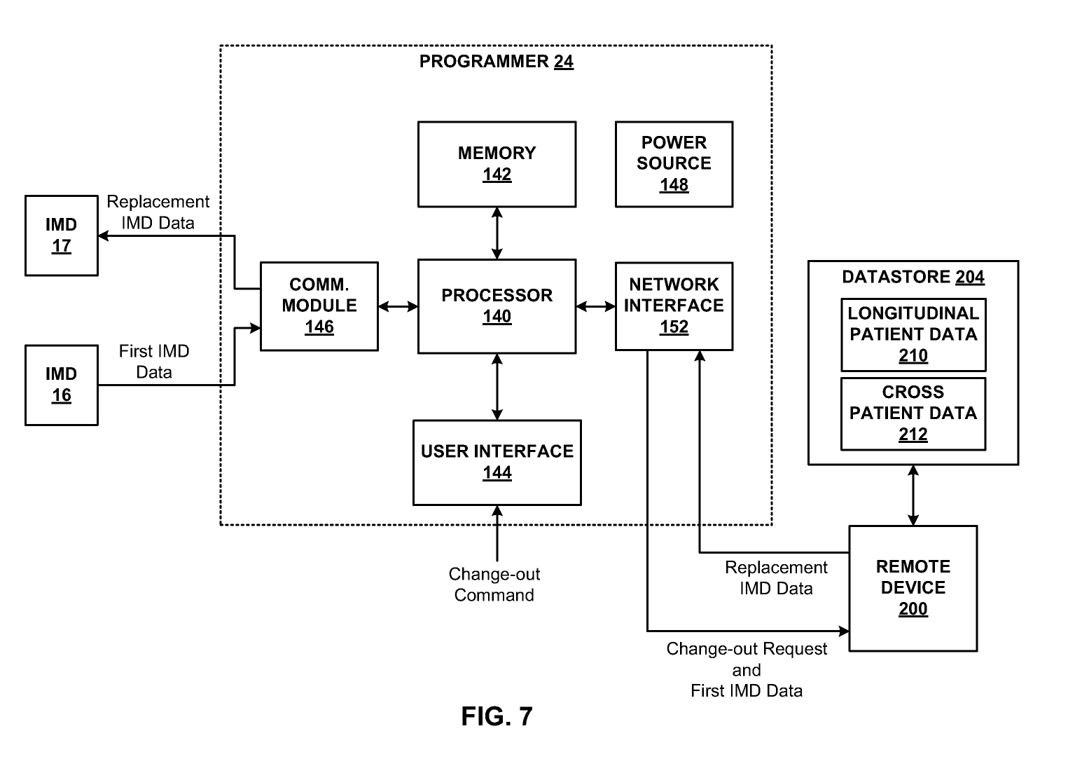

Referring now to FIG. 7, detailed views of an example programmer 24 and example datastore 204 are shown. FIG. 7 illustrates transmission of the first IMD data to programmer 24, receipt of a change-out command from the user, and determination of the replacement IMD data by remote device 200 based on the first IMD data and data from datastore 204.

The clinician may enter a change-out command into user interface 144 of programmer 24 at the initiation of the change-out procedure. For example, the clinician may use the keyboard, mouse, etc, to enter the change-out command. As used herein, the change-out command may represent any single command or sequence of commands used by the clinician to retrieve the first IMD data from first IMD 16, determine the replacement IMD data, and program replacement IMD 17 using the replacement IMD data.

Although replacement IMD data may be determined during the change-out procedure, in some examples the replacement IMD data may be determined prior to the change-out procedure and stored, for example, in datastore 204 for subsequent retrieval. In this example, stored replacement IMD data may be retrieved in response to the change-out command during the change-out procedure.

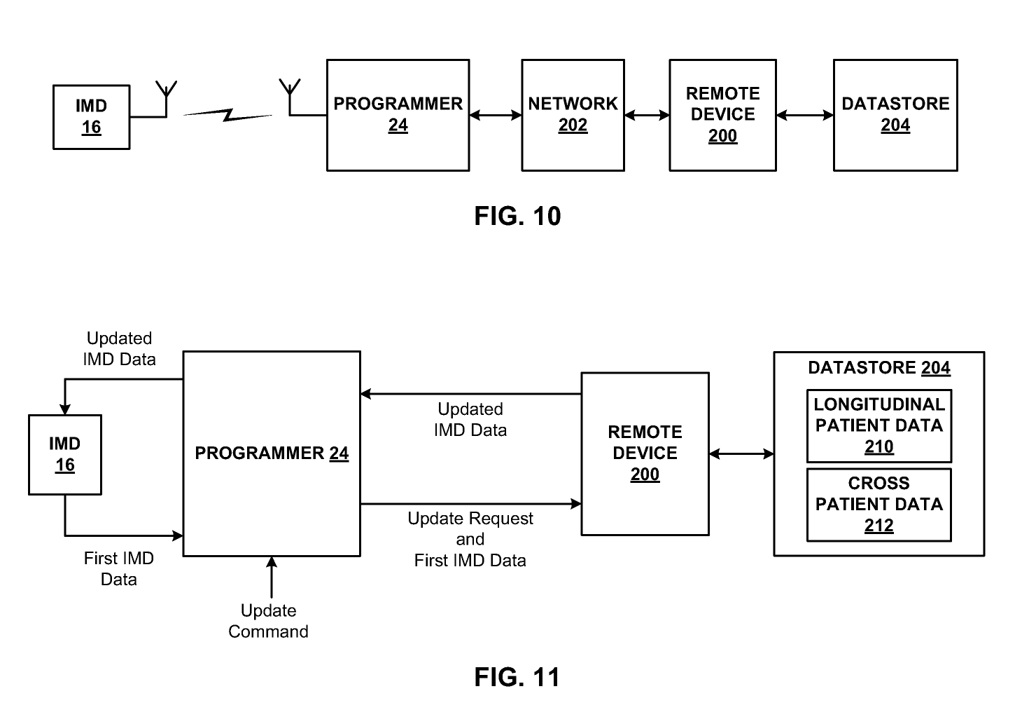

Although FIGS. 6-9 are directed toward determining replacement IMD data for a replacement IMD during a change-out procedure, the techniques of the present disclosure may be generally applicable to updating IMD data based on at least one of longitudinal patient data, cross-patient data, and other medical records. Generally updating data of IMD 16, i.e., not during a change-out, based on at least one of longitudinal patient data, cross-patient data, and other medical records is described herein with reference to FIGS. 10-13.

Programmer 24 may retrieve the first IMD data from first IMD 16 in response to the change-out command. Programmer 24 may then store the first IMD data in memory 142 in response to the change-out command. Specifically, in response to the change-out command, communication module 146 may retrieve the first IMD data from first IMD 16, then processor 140 may store the first IMD data in memory 142.

Programmer 24 may then send a change-out request to remote device 200 indicating that a change-out procedure is in progress. The change-out request may include similar data (e.g., be the same as) and/or be based on the change-out command. Along with the change-out request, programmer 24 may also send the first IMD data to remote device 200, via network 202. Remote device 200 may retrieve data from datastore 204 in response to the change-out request. For example, remote device 200 may retrieve at least one of longitudinal patient data 210, cross-patient data 212, or other medical records. Remote device 200 may then determine the replacement IMD data based on the first IMD data and the data retrieved from datastore 204. The replacement IMD data determined by remote device 200 may include, but is not limited to, alert thresholds, detection intervals, an NID parameter, SVT and/or VT templates, and atrial fibrillation (AF) characteristics. Replacement data may also include updated algorithms, such as, modified EGM comparison algorithms, modified atrial fibrillation detection algorithms, and modified dynamic discrimination algorithms.

Although replacement IMD data may be determined in response to a change-out request based on the change-out command received from the clinician, the replacement IMD data may be generated in other ways. For example, programmer 24 may send a change-out request to remote device 200 at predetermined times, e.g., on predetermined dates or at predetermined intervals in order to initiate a determination of replacement IMD data for storage, e.g., in datastore 204. In other examples, a change-out request may not be sent from programmer 24, but instead generated at remote device 200 according to predetermined times, e.g., on predetermined dates or at predetermined intervals, in order to initiate a determination of replacement IMD data for storage. In still other examples, remote device 200 may initiate the determination of replacement IMD data based on occurrence of a specified event, such as receipt of a predetermined number (e.g., 10) of transmissions. In still other examples, first IMD 16 implanted in patient 14 may generate an indicator that is transmitted to remote device 200 that initiates determination of replacement IMD data. The indicator may be based on remaining battery life, for example.

In summary, remote device 200 may determine the replacement IMD data based on the first IMD data and the data retrieved from datastore 204. Specifically, remote device 200 may determine the replacement IMD data based on at least one of the first IMD data, longitudinal patient data 210, or cross-patient data 212. In some examples, remote device 200 may determine various parameters for transfer to replacement IMD 17 based on at least one of the first IMD data, longitudinal patient data 210, or cross-patient data 212. The various parameters include, but are not limited to, alert thresholds, detection intervals, and NID parameters. Programmer 24 may then transfer the determined parameters to replacement IMD 17. In some implementations, programmer 24 may display the replacement IMD data to the clinician on the display of programmer 24 for the clinician to review. After reviewing the replacement IMD data on the display of programmer 24, the clinician may transfer the replacement IMD data to replacement IMD 17, e.g., by pressing a program button.

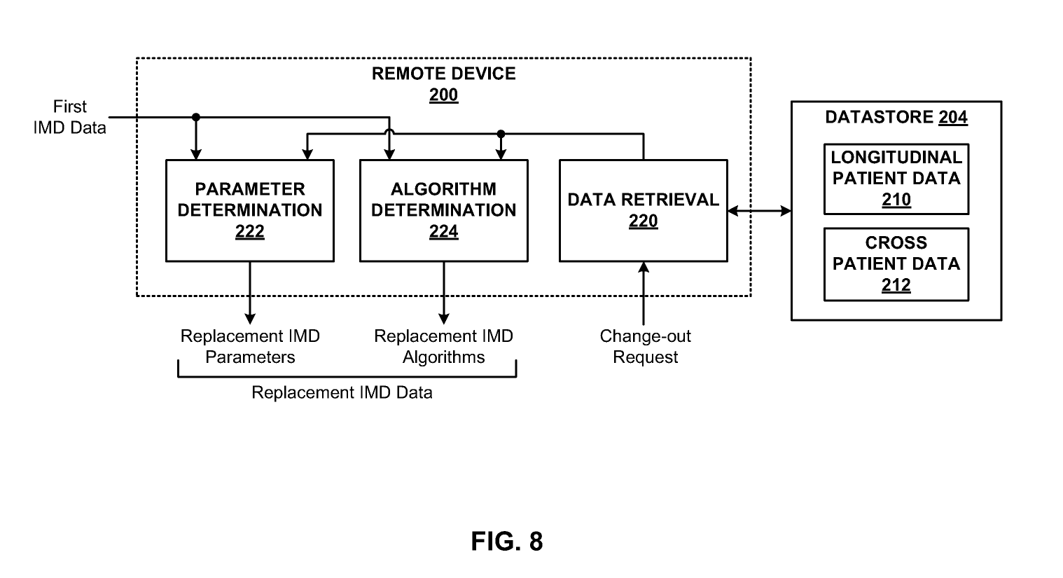

In some examples, remote device 200 may determine various algorithms for transfer to replacement IMD 17 based on at least one of the first IMD data, longitudinal patient data 210, or cross-patient data 212. In other words, remote device 200 may determine that replacement IMD 17 may operate more effectively based on a different algorithm than that used by first IMD 16, and accordingly, may adjust a real-time algorithm that was used in first IMD 16 based on longitudinal data 210 and/or cross-patient data 212. Determination of various parameters and algorithms for transfer to replacement IMD 17 is further discussed hereinafter with reference to FIG. 8. In some aspects, algorithms may be viewed as the various logical functions performed by an IMD, while parameters may be viewed as values that are set within the IMD in order to adjust the operation of the algorithms included in the IMD.

FIG. 8 shows an example implementation of remote device 200. Remote device 200 includes a data retrieval module 220, a parameter determination module 222, and an algorithm determination module 224. Parameter determination module 222 and algorithm determination module 224 may be collectively referred to as a “determination module.” Data retrieval module 220 retrieves data from datastore 204 in response to the change-out request. Parameter determination module 222 and/or algorithm determination module 224 represent the functionality of remote device 200 that determines the replacement IMD data based on the data retrieved from datastore 204 and the first IMD data.

Remote device 200, and modules included in remote device 200, may be implemented, at least in part, in hardware, software, firmware or any combination thereof. For example, various aspects of remote device 200 may be implemented within one or more processors, including one or more microprocessors, DSPs, ASICs, FPGAs, or any other equivalent integrated or discrete logic circuitry, as well as any combinations of such components. When implemented in software, the functionality ascribed to remote device 200 may be embodied as instructions on a computer-readable medium such as RAM, ROM, NVRAM, EEPROM, FLASH memory, magnetic data storage media, optical data storage media, or the like. The instructions may be executed to support one or more aspects of the functionality of remote device 200 described in this disclosure.

Although determination of the replacement IMD data is described as being performed by remote device 200, in some examples, programmer 24 may determine the replacement IMD data. For example, the functionality of remote device 200 as described herein, i.e., modules of remote device 200, may alternatively be implemented in processor 140 of programmer 24 instead of remote device 200. Accordingly, in some examples, programmer 24 may retrieve the data from datastore 204 and the first IMD data from first IMD 16, then determine replacement IMD data based on the first IMD data and data retrieved from datastore 204.

Data retrieval module 220 may retrieve data from datastore 204 in response to the change-out request. For example, data retrieval module 220 may retrieve at least one of longitudinal patient data 210 or cross-patient data 212 from datastore 204 in response to the change-out request. Algorithm determination module 224 may determine replacement IMD algorithms based on the data retrieved by data retrieval module 220 and the first IMD data. Replacement IMD algorithms may represent algorithms to be programmed into replacement IMD 17 based on the first IMD data and the data retrieved from datastore 204. The algorithms, for example, may include instructions that are transferred to memory of replacement IMD 17 and which are executed by a processor of replacement IMD 17 to manipulate other data stored in memory of replacement IMD 17 and to control replacement IMD 17. In other examples, algorithm determination module 224 may enable/disable algorithms in replacement IMD 17 instead of determining replacement IMD algorithms.

Parameter determination module 222 may determine replacement IMD parameters based on the data retrieved by data retrieval module 220 and the first IMD data. The replacement IMD parameters may include data that may be transferred to replacement IMD 17 other than algorithms. Accordingly, the combination of the replacement IMD parameters and the replacement IMD algorithms may be referred to collectively as the “replacement IMD data.” As described herein, the replacement IMD parameters that may be determined by parameter determination module 222 may include, but are not limited to, alert thresholds, detection intervals, NID parameters, SVT and/or VT templates, and AF characteristics. As described herein, replacement IMD algorithms that may be determined by algorithm determination module 224 may include, but are not limited to, EGM comparison algorithms, tachyarrhythmia (e.g., VF, VT, SVT or AF) detection algorithms, and dynamic cardiac rhythm discrimination algorithms, e.g., tachyarrhythmia discrimination algorithms, such as to discriminate ventricular tachyarrhythmia from supraventricular tachyarrhythmia. Determination of replacement IMD parameters and replacement IMD algorithms are now discussed in turn.

Parameter determination module 222 may determine updated alert thresholds (e.g., impedance alert thresholds) for replacement IMD 17 when alert thresholds of first IMD 16 are not optimal, as suggested by longitudinal patient data 210, cross-patient data 212, and other medical records. As a device lead matures in a chronic implant, the impedance properties of that lead may change over time. Accordingly, in some examples, alert thresholds may diverge from optimal values due to aging of device leads. Without adjustment of lead impedance alert thresholds, a straight copy of the original threshold from first IMD 16 to replacement IMD 17 may lead to less meaningful alerts since the older alert threshold setting may not account for the lead maturation as characterized by the longitudinal data. In some examples, cross-patient data may include a large data set on long term use of particular leads across a large population. In these examples, parameter determination module 222 may use cross-patient data to characterize an impedance maturation trend for particular lead models. This integration of information generated by the use of certain lead models by a large number of patients over a period of time may be used to define a new operating point of leads currently implanted in patient 14 at the point in time (e.g., change-out procedure) pertinent to setting of the alert threshold, thereby impacting the clinically appropriate impedance threshold that should be set for alerting the clinician.

In the case of lead impedances, parameter determination module 222 may determine that lead impedance alert thresholds of the first IMD data are not optimal as compared to alert thresholds suggested by longitudinal patient data 210 when alert thresholds of the first IMD data differ from alert thresholds (e.g., by a predetermined amount) suggested by longitudinal patient data 210. Specifically, in one example, if the first IMD data includes an alert threshold of 1500 ohms, but parameter determination module 222 determines that typical lead impedance for patient 14 is approximately 700 ohms based on longitudinal patient data 210, parameter determination module 222 may determine that alert thresholds for replacement IMD 17 should be set to a value of less than 1500 ohms but greater than 700 ohms, e.g., 1000 ohms. Accordingly, parameter determination module 222 may set alert thresholds to 1000 ohms based on the example longitudinal patient data 210 and the example first IMD data. Programmer 24 may program replacement IMD 17 with the 1000 ohm impedance threshold based on the replacement IMD data received from remote device 200, thus, personalizing impedance thresholds of replacement IMD 17 for patient 14 based on longitudinal patient data 210.

Parameter determination module 222 may also modify alert thresholds associated with AF burden (and ventricular response during AF) based on longitudinal patient data 210 and/or cross-patient data 212 from patients of the same cohort. Parameter determination module 222 may determine a typical AF burden for patient 14 based on longitudinal patient data 210 and cross-patient data 212. If the AF burden threshold of the first IMD data differs (e.g., by a predetermined amount of hours) from an appropriate AF burden as determined based on a modeling of patient's own longitudinal patient data 210 and data from the patients having the same cohort, then parameter determination module 222 may set the AF burden threshold to a value that is more appropriate. Accordingly, parameter determination module 222 may set the AF burden threshold based on longitudinal patient data 210 and the first IMD data. In some examples, parameter determination module 222 may set the AF burden threshold based on cross-patient data including patients having the same cohort, e.g., based on indications of which AF burden threshold may be the most effective amongst these patients. For example, such cross-patient data may indicate that first IMD data does not include an appropriate threshold since such a threshold in the patients having a similar cohort may be associated with poor clinical outcomes. Programmer 24 may program replacement IMD 17 with the AF burden threshold based on the replacement IMD data received from remote device 200, thus, personalizing the AF burden threshold of replacement IMD 17 for patient 14.

In one example, the value of having longitudinal and cross-patient data available for determination of replacement IMD data is that the longitudinal data and cross-patient data may enable setting of an appropriate threshold for AF burden that may be out of character for a particular patient. Specifically, if patient 14 had usually experienced less than 4 hours of AF per day, and this stays the same over time, then replacement IMD data may be the same as first IMD data. But, if patient 14 had recently experienced a greater or lesser amount of AF burden as evidenced by longitudinal data, then replacement IMD data may be generated that includes a modified AF burden threshold. In this manner, alerts to a clinician indicating that a patient is experiencing a clinically relevant change in AF burden may be tailored to the particular patient based on longitudinal patient data.

Other diagnostic alert thresholds such as fluid status monitoring (e.g., OptiVol® available via Carelink®) and patient activity monitoring could benefit from a similar approach to the alert threshold modifications as described above. For example, parameter settings in replacement IMD data for the fluid status monitoring (, e.g., a threshold for a fluid index) could be set based on longitudinal data 210 since longitudinal data 210 may indicate when the fluid index enters a critical zone for the patient.

Parameter determination module 222 may also modify alert thresholds associated with episode frequency. Parameter determination module 222 may determine an appropriate episode frequency alert threshold for patient 14 based on longitudinal patient data 210 and/or cross-patient data 212. If the episode frequency threshold of the first IMD data differs (e.g., by a predetermined amount) from the appropriate episode frequency threshold as determined based on longitudinal patient data 210 and/or cross patient data 212, then parameter determination module 222 may set the episode frequency threshold to a value that is more appropriate, i.e., a value that is more personalized and meaningful to the patient. Accordingly, parameter determination module 222 may set the episode frequency threshold based on longitudinal patient data 210 and the first IMD data. Programmer 24 may program replacement IMD 17 with the episode frequency threshold based on the replacement IMD data received from remote device 200, thus, personalizing the episode frequency threshold of replacement IMD 17 for patient 14. For example, first IMD 16 may have a threshold relating to frequency of AF episodes, e.g., per day, which may be adjusted in the programming of replacement IMD 17 with replacement IMD data in this manner.

Parameter determination module 222 may also modify alert thresholds associated with frequency of other events. For example, parameter determination module may modify an alert threshold associated with frequency of mode switching by an IMD. Parameter determination module 222 may determine an appropriate mode-switch frequency threshold for alerting for patient 14 based on longitudinal patient data 210 and/or cross-patient data 212. If the frequency threshold of the first IMD data differs (e.g., by a predetermined amount) from the appropriate episode frequency as determined based on longitudinal patient data 210 and/or cross patient data 212, then parameter determination module 222 may set the frequency threshold to a value that is more appropriate.

Parameter determination module 222 may also modify detection intervals, e.g., tachycardia detection intervals (TDI) and fibrillation detection intervals (FDI), based on longitudinal patient data 210. For example, parameter determination module 222 may determine that detection intervals of the first IMD data are not optimal as compared to detection intervals suggested by longitudinal patient data 210 when detection intervals of the first IMD data differ from detection intervals suggested by longitudinal patient data 210 by a predetermined amount. Specifically, in one example, if the first IMD data includes a TDI that is set to 150 beats per minute, but longitudinal patient data 210 (e.g., several years of data) suggest that patient 14 has SVTs from 140-150 bpm and true VTs at rates above 160 bpm, parameter determination module 222 may set TDI at 160 bpm in replacement IMD data. Accordingly, programmer 24 may program replacement IMD 17 with TDI set to 160 bpm based on the first IMD data and longitudinal patient data 210, thus, personalizing TDI parameters of replacement IMD 17 for patient 14.

Parameter determination module 222 may modify NID parameters based on longitudinal patient data 210. For example, parameter determination module 222 may determine that NID parameters of the first IMD data are not optimal as compared to NID parameters suggested by longitudinal patient data 210 when NID parameters of the first IMD data differ from NID parameters suggested by longitudinal patient data 210 by a predetermined amount. Specifically, in one example, if the first IMD data includes NID parameters set to 12/16, but longitudinal patient data 210 suggests that several episodes (e.g., VT) of patient 14 are self terminating after diagnosis and before delivery of therapy, then parameter determination module 222 may extend NID, for example, to 18/24. In other words, if longitudinal patient data 210 indicates that episodes of patient 14 self terminate, then it may be beneficial to extend NID (e.g., to 18/24) to more readily allow an episode to self terminate without application of therapy. Accordingly, programmer 24 may program replacement IMD 17 with an extended NID based on the first IMD data and longitudinal patient data 210, thus, personalizing NID parameters of replacement IMD 17 for patient 14. Although NID parameters listed above include a ratio (e.g., 18/24) that may represent a percentage of consecutive cardiac intervals shorter than a threshold and be applicable to NID for VF, in other examples, a NID parameter, e.g., for VT, may include only a single number, e.g., 12 consecutive intervals shorter than a threshold.

Furthermore, parameter determination module 222 may modify NID parameters of the first IMD data based on an effect the NID parameters may have on patient 14. For example, the first IMD data may not be optimal for replacement IMD 17 if parameter determination module 222 determines that longitudinal patient data 210 indicates that patient 14 experiences syncope at the current NID. Parameter determination module 222 may determine whether patient 14 experiences syncope at the NID of the first IMD data based on longitudinal patient data such as activity, heart rate, intrathoracic or intracardiac pressure, or respiration rate. If parameter determination module 222 determines that longitudinal patient data 210 indicates that patient 14 experiences syncope at the current NID, parameter determination module 222 may shorten NID. Accordingly, programmer 24 may program replacement IMD 17 with a shortened NID based on the first IMD data and longitudinal patient data 210, thus, personalizing NID parameters of replacement IMD 17 for patient 14.

Parameter determination module 222 may modify pending values for an SVT and/or VT template, used for real-time wavelet analysis, for patient 14 based on longitudinal patient data 14. Parameter determination module 222 may compare the SVT and VT template of the first IMD data with an SVT and VT template, respectively, determined based on the longitudinal patient data 210. If parameter determination module 222 determines that the SVT and/or VT templates based on the longitudinal patient data 210 differ from the first IMD data, then parameter determination module 222 may recommend that the SVT and/or VT templates be updated. For example, parameter determination module 222 may determine an updated SVT and/or VT template from EGMs in longitudinal data 210, then compare the updated templates with currently used templates to determine whether the current templates are optimal. If the current templates are not optimal, parameter determination module 222 may recommend that the templates be updated. The clinician may review the recommended SVT and/or VT templates on programmer 24 to determine whether to upload the templates to replacement IMD 17.

Parameter determination module 222 may, based on analysis of longitudinal patient data 210, recommend modification to therapy parameter settings that control delivery of ATP. For example, parameter determination module 222 may determine that longitudinal patient data 210 suggests that ATP is more effective in treating patient VT episodes when various characteristics are present in the VT episodes. Characteristics may include: whether the episode is monomorphic or polymorphic, a cycle length of the episode, a variability in cycle length during the episode, characteristics of the onset of the episode (e.g., a rate of onset), stability during the episode, a specific morphology of the episode, an atrioventricular relationship during the episode, etc. Parameter determination module 222 may analyze longitudinal patient data 210 to determine for which characteristics ATP was effective, then parameter determination module 222 may recommend changes to therapy parameter settings based on the analysis. The clinician may review the recommended changes to the therapy parameter settings to determine whether to upload the therapy parameter settings to replacement IMD 17.

The ability of remote device 200, based on data stored in datastore 204, to be able to determine the type of VT (e.g., cycle length and morphology) that is successfully terminated with ATP, along with the parameters for the ATP delivery (e.g., number of pulses) that successfully terminated the VT may allow for a more effective modification to therapy parameter settings that control delivery of ATP. Although VT properties may be specific to a patient, in some examples, based on cross-patient data 212 of the same cohort as patient 14, remote device 200 may optimize ATP more effectively, e.g., by setting maximum range settings associated with the cross-patient data 212.

Parameter determination module 222 may, based on analysis of longitudinal patient data 210, recommend modification to defibrillation pulse strengths. For example, parameter determination module 222 may determine that longitudinal patient data 210 suggests that electrical pulses of a particular strength are more effective in treating patient VT/VF episodes when various characteristics are present in the VT/VF episodes. Characteristics corresponding to VT or VF may include: whether the episode is monomorphic or polymorphic, a cycle length of the episode, a variability in cycle length during the episode, characteristics of the onset of the episode (e.g., a rate of onset), stability during the episode, a specific morphology of the episode, an atrioventricular relationship during the episode, etc. Parameter determination module 222 may analyze longitudinal patient data 210 to determine which pulse strengths were effective in treating the patient based on episode characteristics, then parameter determination module 222 may recommend changes to settings of defibrillation pulse strengths based on the analysis. The clinician may review the recommended changes to the settings which control defibrillation pulse strengths to determine whether to upload the settings to replacement IMD 17.

In other examples, parameter determination module 222 may, based on analysis of at least one of respiration, fluid retention, or thoracic impedance, provide recommendations for settings of the IMD relating to heart failure, e.g., settings that control warnings related to heart failure.

In some examples, parameter determination module 222 may provide recommendations for parameter settings of the IMD relating to rate response based on analysis of at least one of activity threshold, activity acceleration, activity deceleration, and rate response slope. Such rate response parameters may benefit from optimizations that serve to provide the optimal pacing therapy to a patient during changes in activity level, e.g., sitting vs. walking Modifications to rate response parameters may leverage data such as past patient activity, age, and rate response parameters of first IMD data to determine the rate response parameters of the replacement IMD data. In some cases, comparison of rate response data to cross patient data 212 of similar patients may assist in determining if rate response settings in the replacement IMD data may be inappropriate. e.g., too responsive, or not responsive enough.

In some implementations, parameter determination module 222 may be used to determine whether to even implement a certain parameter in a newly implanted IMD based on cross-patient data 212 when the patient has not had an IMD implanted in the past. For example, for a patient receiving a new IMD, parameter determination module 222 may determine whether a certain parameter may be effective when implemented in the IMD based on whether the parameter was effective in segments of the population having similar characteristics as the new patient.

Algorithm determination module 224 may modify various algorithms to be programmed into replacement IMD 17. For example, algorithm determination module 224 may modify EGM comparison algorithms. In one example, if an arrhythmia detection algorithm of first IMD 16 compares a current EGM to a single waveform template, but algorithm determination module 224 determines, based on longitudinal patient data 210, that replacement IMD 17 may perform more effectively if replacement IMD 17 compares the current waveform to multiple templates, algorithm determination module 224 may modify the EGM comparison algorithm to compare the current waveform to multiple templates. Programmer 24 may then transfer the modified algorithm to replacement IMD 17.

Algorithm determination module 224 may further modify EGM comparison algorithms based on longitudinal data 210 relating to cardiac cycle length. For example, based on longitudinal data 210, algorithm determination module 224 may determine whether patient 14 exhibits certain EGMs that correspond to certain cycle lengths. If patient 14 exhibits certain EGMs that correspond to certain cycle lengths, then algorithm determination module 224 may modify the EGM comparison algorithm of first IMD 16 to compare the waveforms to certain templates (e.g., VT and/or SVT templates) based on a corresponding cycle length. Accordingly, algorithm determination module 224 may modify the EGM comparison algorithm to select different comparison templates based on longitudinal data 210. Subsequently, algorithm determination module 224 may transfer the modified EGM comparison algorithm to replacement IMD 17.

In a similar manner, algorithm determination module 224 may modify other algorithms that algorithm determination module 224 determines, based on longitudinal patient data 210, to be more effective than algorithms that are currently implemented in first IMD 16. For example, algorithm determination module 224 may modify atrial fibrillation detection algorithms. As a further example, algorithm determination module 224 may determine whether dynamic tachyarrhythmia discrimination algorithms are optimal for patient 14 based on longitudinal patient data 210. Dynamic tachyarrhythmia discrimination algorithms, which may include adding pacing into an arrhythmia to determine an origin of the arrhythmia, may be more effective in some patients, and the determination of whether or not to implement dynamic discrimination algorithms in replacement IMD 17 may be made by algorithm determination module 224 based on longitudinal patient data 210.

In some examples, algorithm determination module 224 may determine whether to implement an algorithm in replacement IMD 17 based on cross-patient data 212. For example, algorithm determination module 224 may determine that a certain algorithm may be more effective with patient 14 if the certain algorithm is effective in certain segments of the population having similar characteristics as patient 14, as indicated by cross-patient data 212. Algorithm determination module 224 may therefore suggest, based on cross-patient data 212, using the certain algorithm in replacement IMD 17 when the certain algorithm is found to be more effective in segments of the population that have similar characteristics as patient 14. For example, algorithm determination module 224 may determine that a segment of the population has similar characteristics as patient 14 if the population has similar indications, comorbidities, similar frequencies of therapies delivered, similar types of arrhythmias present, or similar types of leads implanted.

In other examples, parameter determination module 222 and/or algorithm determination module 224 may, based on analysis of longitudinal data 210, provide recommendations for arrhythmia prevention algorithm settings or behavior based on analysis. For example, parameter determination module 222 may analyze flashback memory data within longitudinal data 210 to predict when VT might occur, and then make recommendations for arrhythmia prevention settings based on the analysis.

In some examples, parameter determination module 222 and/or algorithm determination module 224 may determine which detection vector (i.e., which electrodes and leads) provided the best historical performance with regard to detection of cardiac events, e.g., depolarizations, based on longitudinal patient data 210. Based on the determination, modules 222, 224 may generate replacement IMD data that configures replacement IMD 17 to use the best determined configuration.

In still other examples, parameter determination module 222 and/or algorithm determination module 224 may provide recommendations for changes to parameters or real-time algorithm behavior of replacement IMD 17 based on analysis of data such as night and day heart rate. For example, day and night heart rate data collected by IMD 16 and stored in datastore 204 may be used to set upper and lower heart rates used by IMD 17 to determine when the heart rate of patient 14 is out of an expected range at a particular time of day. The upper and lower heart rates may be used by IMD 17, for example, to alert a clinician that a patient's nighttime heart rate is greater than an expected maximum.

In some implementations, algorithm determination module 224 may be used to determine whether to implement a certain algorithm in a newly implanted IMD based on cross-patient data 212 when the patient has not had an IMD implanted in the past. For example, for a patient receiving a new IMD, algorithm determination module 224 may be used to determine whether a certain algorithm may be effective with the patient based on whether the certain algorithm is effective in segments of the population having similar characteristics as the new patient.

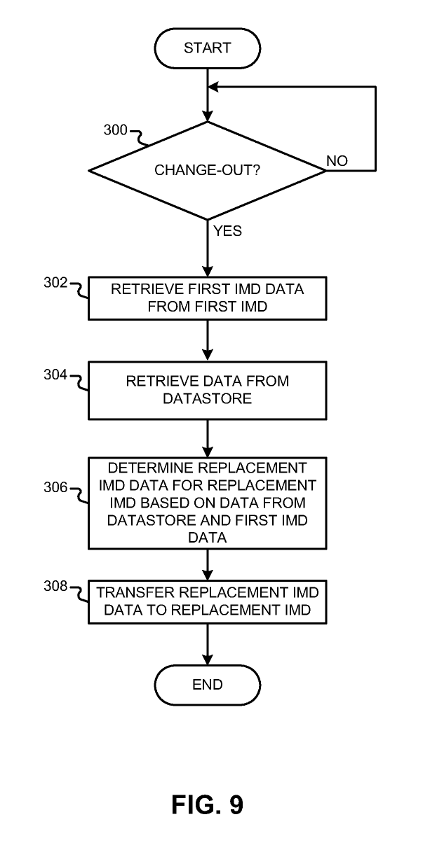

Referring now to FIG. 9, a method for determining replacement IMD data during a change-out procedure is shown. Programmer 24 determines whether a change-out procedure is requested by the clinician (300). For example, the clinician may enter a change-out command in user interface 144 of programmer 24 to initiate a change-out procedure. Accordingly, programmer 24 may determine that a change-out is requested by the clinician upon receiving the change-out command. Programmer 24 retrieves first IMD data from first IMD 16 in response to the change-out command (302). Remote device 200 retrieves data from datastore 204 (304). For example, data retrieved from datastore 204 may include longitudinal patient data 210 and/or cross-patient data 212. Remote device 200 determines replacement IMD data based on the data retrieved from datastore 204 and the first IMD data received from programmer 24 (306). Specifically, remote device 200 may determine the replacement IMD data based on at least one of the first IMD data, longitudinal patient data 210, or cross-patient data 212. Replacement data may include replacement IMD parameters and/or replacement IMD algorithms determined by remote device 200. Programmer 24 transfers the replacement IMD data to replacement IMD 17 (308).

Although FIGS. 6-9 and the above description are directed toward determining replacement IMD data for a replacement IMD during a change-out procedure, the techniques described above may be generally applicable to updating IMD data, e.g., not during a change-out procedure, based on at least one of longitudinal patient data and cross-patient data. Generally updating data of IMD 16 based on at least one of longitudinal patient data and cross-patient data is described herein with reference to FIGS. 10-13.

FIG. 10 illustrates communication between (e.g., transmission of data between) IMD 16 and programmer 24 during an update procedure. In FIG. 10, parameters and algorithms of IMD 16 are updated during the update procedure. Data retrieved from IMD 16 during or before the update procedure may be referred to as “first IMD data.” Data transmitted to IMD 16 during the update procedure may be referred to as “updated IMD data.” The updated IMD data may be determined based on the first IMD data, and in some cases also based on data retrieved from datastore 204. For example, programmer 24 and/or remote device 200 may determine updated IMD data based on the first IMD data and the data retrieved from datastore 204, e.g., data associated with patient 14 and/or other patients.

Although transmission of data from first IMD 16 to programmer 24 may occur during the update procedure, in other examples, transmission of data from first IMD 16 may occur at a time prior to the update procedure. For example, data from first IMD 16 may be transmitted to datastore 204 prior to the update procedure and then subsequently used during the update procedure, e.g., retrieved from datastore 204 in response to a command entered during the update procedure.

Based on data retrieved from datastore 204 and the first IMD data retrieved from first IMD 16, remote device 200 may determine the updated IMD data for IMD 16. Remote device 200 may then send the updated IMD data to programmer 24 for transmission to replacement IMD 16 during the update procedure. Accordingly, during the update procedure, IMD 16 may be programmed with updated IMD data that is based on at least one of the first IMD data, the longitudinal patient data, or the cross-patient data. Although the present disclosure describes remote device 200 as determining the updated IMD data based on the first IMD data, the longitudinal data, and/or the cross-patient data, in other implementations, programmer 24 and/or remote device 200, alone or in combination, may determine the updated IMD data.

FIG. 11 illustrates transmission of the first IMD data to programmer 24, receipt of an update command from the user, and determination of the updated IMD data by remote device 200 based on the first IMD data and data from datastore 204.

The clinician may enter an update command into user interface 144 of programmer 24 at the initiation of the update procedure. For example, the clinician may use the keyboard, mouse, etc, to enter the update command. As used herein, the update command may represent any single command or sequence of commands used by the clinician to retrieve the first IMD data from first IMD 16, determine the updated IMD data, and program IMD 16 using the updated IMD data.

Although updated IMD data may be determined during the update procedure, in some examples the updated IMD data may be determined prior to the update procedure and stored, for example, in datastore 204 for subsequent retrieval. In this example, stored updated IMD data may be retrieved in response to the update command during the update procedure.

Programmer 24 may retrieve the first IMD data from first IMD 16 in response to the update command. Programmer 24 may then store the first IMD data in memory 142 in response to the update command. Specifically, in response to the update command, communication module 146 may retrieve the first IMD data from first IMD 16, then processor 140 may store the first IMD data in memory 142.

Programmer 24 may then send an update request to remote device 200 indicating that an update procedure is in progress. The update request may include similar data (e.g., be the same as) and/or be based on the update command. Along with the update request, programmer 24 may also send the first IMD data to remote device 200, via network 202. Remote device 200 may retrieve data from datastore 204 in response to the update request. For example, remote device 200 may retrieve at least one of longitudinal patient data 210 or cross-patient data 212. Remote device 200 may then determine the updated IMD data based on the first IMD data and the data retrieved from datastore 204. The updated IMD data determined by remote device 200 may include, but is not limited to, alert thresholds, detection intervals, an NID parameter, SVT and/or VT templates, and atrial fibrillation (AF) characteristics. Updated IMD data may also include updated algorithms, such as, modified EGM comparison algorithms, modified atrial fibrillation detection algorithms, and modified dynamic discrimination algorithms.

Although the update request is described as being based on the update command received from the clinician, the update request may be generated in other ways. For example, programmer 24 may send an update request to remote device 200 at predetermined times, e.g., on predetermined dates or at predetermined intervals in order to initiate an update procedure. In other examples, an update request may not be sent from programmer 24, but instead generated at remote device 200 according to predetermined times, e.g., on predetermined dates or at predetermined intervals. In still other examples, remote device 200 may initiate the update procedure based on occurrence of a specified event, such as receipt of a predetermined number (e.g., 10) of transmissions. In other examples, IMD 16 implanted in patient 14 may generate an indicator that is transmitted to remote device 200 that initiates determination of updated IMD data. The indicator may be based on remaining battery life, for example.

In summary, remote device 200 may determine the updated IMD data based on the first IMD data and the data retrieved from datastore 204. Specifically, remote device 200 may determine the updated IMD data based on at least one of the first IMD data, longitudinal patient data 210, or cross-patient data 212. In some examples, remote device 200 may determine various parameters for transfer to IMD 16 based on at least one of the first IMD data, longitudinal patient data 210, or cross-patient data 212. The various parameters include, but are not limited to, alert thresholds, detection intervals, and NID parameters. Programmer 24 may then transfer the determined parameters to IMD 16. In some implementations, programmer 24 may display the updated IMD data to the clinician on the display of programmer 24 for the clinician to review. After reviewing the updated IMD data on the display of programmer 24, the clinician may transfer the updated IMD data to IMD 16, e.g., by pressing a program button.

In some examples, remote device 200 may determine various algorithms for transfer to IMD 16 based on at least one of the first IMD data, longitudinal patient data 210, or cross-patient data 212. In other words, remote device 200 may determine that IMD 16 may operate more effectively based on a different algorithm than is currently used by IMD 16, and accordingly, may adjust a real-time algorithm that is currently used in IMD 16 based on longitudinal data 210 and/or cross-patient data 212. Determination of various parameters and algorithms for transfer to IMD 16 is discussed further hereinafter.

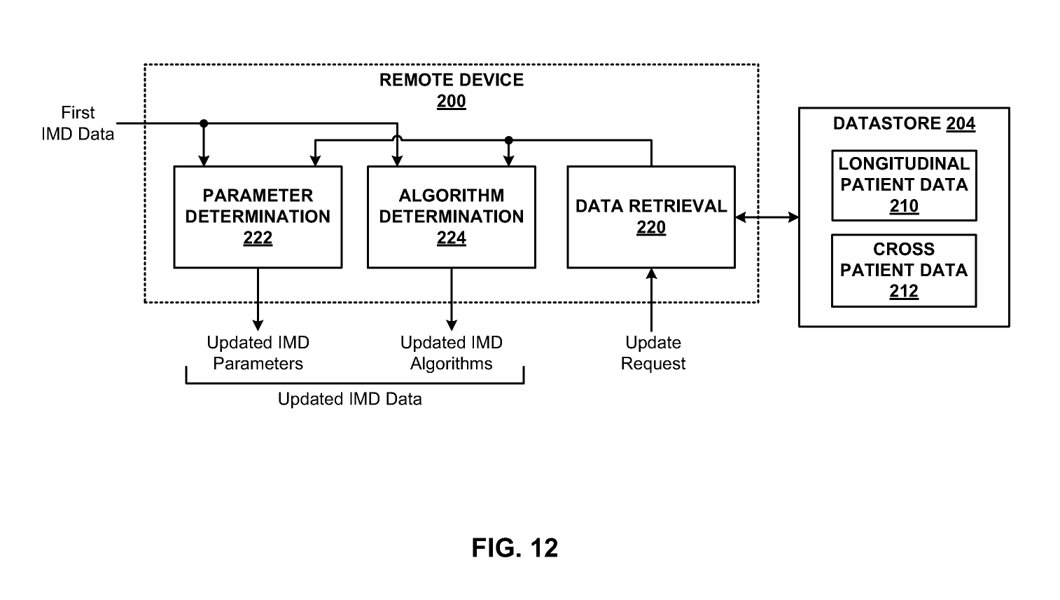

FIG. 12 shows an example implementation of remote device 200. Data retrieval module 220 retrieves data from datastore 204 in response to the update request. Parameter determination module 222 and/or algorithm determination module 224 represent the functionality of remote device 200 that determines the updated IMD data based on the data retrieved from datastore 204 and the first IMD data.

Although determination of the updated IMD data is described as being performed by remote device 200, in some examples, programmer 24 may determine the updated IMD data. For example, the functionality of remote device 200 as described herein, i.e., modules of remote device 200, may alternatively be implemented in processor 140 of programmer 24 instead of remote device 200. Accordingly, in some examples, programmer 24 may retrieve the data from datastore 204 and the first IMD data from IMD 16, then determine updated IMD data based on the first IMD data and data retrieved from datastore 204.

Data retrieval module 220 may retrieve data from datastore 204 in response to the update request. For example, data retrieval module 220 may retrieve at least one of longitudinal patient data 210 or cross-patient data 212 from datastore 204 in response to the update request. Algorithm determination module 224 may determine updated IMD algorithms based on the data retrieved by data retrieval module 220 and the first IMD data. Updated IMD algorithms may represent algorithms to be programmed into IMD 16 based on the first IMD data and the data retrieved from datastore 204. The updated IMD algorithms, for example, may include instructions that are transferred to memory of IMD 16 and which are executed by a processor of IMD 16 to manipulate other data stored in memory of IMD 16 and to control IMD 16. In other examples, algorithm determination module 224 may enable/disable algorithms of IMD 16 instead of determining updated IMD algorithms.

Parameter determination module 222 may determine updated IMD parameters based on the data retrieved by data retrieval module 220 and the first IMD data. The updated IMD parameters may include data that may be transferred to IMD 16 other than algorithms. Accordingly, the combination of the updated IMD parameters and the updated IMD algorithms may be referred to collectively as the “updated IMD data.” As described herein, the updated IMD parameters that may be determined by parameter determination module 222 may include, but are not limited to, alert thresholds, detection intervals, NID parameters, SVT and/or VT templates, and AF characteristics. As described herein, updated IMD algorithms that may be determined by algorithm determination module 224 may include, but are not limited to, EGM comparison algorithms, tachyarrhythmia (e.g., VF, VT, SVT or AF) detection algorithms, and dynamic cardiac rhythm discrimination algorithms, e.g., tachyarrhythmia discrimination algorithms, such as to discriminate ventricular tachyarrhythmia from supraventricular tachyarrhythmia. Determination of updated IMD parameters and updated IMD algorithms are now discussed in turn.

Parameter determination module 222 may determine updated alert thresholds (e.g., impedance alert thresholds) for IMD 16 when alert thresholds of IMD 16 are not optimal, as suggested by longitudinal patient data 210, cross-patient data 212, and other medical records. Without adjustment of lead impedance alert thresholds, first IMD data may include less meaningful alerts since the older alert threshold setting may not account for the lead maturation as characterized by the longitudinal data. In some examples, parameter determination module 222 may use cross-patient data to characterize an impedance maturation trend for particular lead models. This integration of information generated by the use of certain lead models by a large number of patients over a period of time may be used to define a new operating point of leads currently implanted in patient 14 at the point in time (e.g., update procedure) pertinent to setting of the alert threshold, thereby impacting the clinically appropriate impedance threshold that should be set for alerting the clinician.

In the case of lead impedance, parameter determination module 222 may determine that current lead impedance alert thresholds of the first IMD data are not optimal in comparison with alert thresholds suggested by longitudinal patient data 210 when alert thresholds of the first IMD data differ from alert thresholds (e.g., by a predetermined amount) suggested by longitudinal patient data 210. Specifically, in one example, if the first IMD data includes an alert threshold of 1500 ohms, but parameter determination module 222 determines that typical lead impedance for patient 14 is approximately 700 ohms based on longitudinal patient data 210, parameter determination module 222 may determine that alert thresholds for IMD 16 should be set to a value of less than 1500 ohms but greater than 700 ohms, e.g., 1000 ohms. Accordingly, parameter determination module 222 may set alert thresholds to 1000 ohms based on the example longitudinal patient data 210 and the example first IMD data. Programmer 24 may program IMD 16 with the 1000 ohm impedance threshold based on the updated IMD data received from remote device 200, thus, updating impedance thresholds of IMD 16 for patient 14 based on longitudinal patient data 210.

Parameter determination module 222 may also modify alert thresholds associated with AF burden (and ventricular response during AF) based on longitudinal patient data 210 and/or cross-patient data 212 from patients of the same cohort. Parameter determination module 222 may determine a typical AF burden for patient 14 based on longitudinal patient data 210 and cross-patient data 212. If the AF burden threshold of the first IMD data differs (e.g., by a predetermined amount of hours) from an appropriate AF burden as determined based on a modeling of patient's own longitudinal patient data 210 and data from the patients having the same cohort, then parameter determination module 222 may set the AF burden threshold of the updated IMD data to a value that is more appropriate. Accordingly, parameter determination module 222 may set the AF burden threshold of the updated IMD data based on longitudinal patient data 210 and the first IMD data. In some examples, parameter determination module 222 may set the AF burden threshold based on cross-patient data including patients having the same cohort, e.g., based on indications of which AF burden threshold may be the most effective amongst these patients. For example, such cross-patient data may indicate that first IMD data does not include an appropriate threshold since such a threshold in the patients having a similar cohort may be associated with poor clinical outcomes. Programmer 24 may program IMD 16 with the AF burden threshold based on the updated IMD data received from remote device 200, thus, updating the AF burden threshold of IMD 16 for patient 14.

In one example, the value of having longitudinal and cross-patient data available for determination of updated IMD data is that the longitudinal data and cross-patient data may enable setting of an appropriate threshold for AF burden that may be out of character for a particular patient. Specifically, if patient 14 had usually experienced less than 4 hours of AF per day, and this stays the same over time, then updated IMD data may be the same as first IMD data. But, if patient 14 had recently experienced a greater or lesser amount of AF burden as evidenced by longitudinal data, then updated IMD data may be generated that includes a modified AF burden threshold. In this manner, alerts to a clinician indicating that a patient is experiencing AF burden may be tailored to the particular patient based on longitudinal patient data.

Other diagnostic alert thresholds such as fluid status monitoring (e.g., OptiVol® available via Carelink®) and patient activity monitoring could benefit from a similar approach to the alert threshold modifications as described above. For example, parameter settings in updated IMD data for the fluid status monitoring (, e.g., a threshold for a fluid index) could be set based on longitudinal data 210 since longitudinal data 210 may indicate when the fluid index enters a critical zone for the patient.

Parameter determination module 222 may also modify current alert thresholds associated with episode frequency. Parameter determination module 222 may determine an appropriate episode frequency alert threshold for patient 14 based on longitudinal patient data 210 and/or cross-patient data 212. If the episode frequency threshold of the first IMD data differs (e.g., by a predetermined amount) from the appropriate episode frequency threshold as determined based on longitudinal patient data 210 and/or cross-patient data 212, then parameter determination module 222 may set the episode frequency threshold of the updated IMD data to a value that is more appropriate. Accordingly, parameter determination module 222 may set the episode frequency threshold of the updated IMD data based on longitudinal patient data 210 and the first IMD data. Programmer 24 may program IMD 16 with the episode frequency threshold based on the updated IMD data received from remote device 200, thus, updating the episode frequency threshold of IMD 16 for patient 14. For example, first IMD 16 may have a threshold relating to frequency of AF episodes, e.g., per day, which may be updated in the reprogramming of IMD 16 with updated IMD data in this manner.

Parameter determination module 222 may also modify current detection intervals, e.g., tachycardia detection intervals (TDI) and fibrillation detection intervals (FDI), based on longitudinal patient data 210. For example, parameter determination module 222 may determine that detection intervals of the first IMD data are not optimal as compared with detection intervals suggested by longitudinal patient data 210 when detection intervals of the first IMD data differ from detection intervals suggested by longitudinal patient data 210 by a predetermined amount. Specifically, in one example, if the first IMD data includes a TDI that is set to 150 beats per minute, but longitudinal patient data 210 (e.g., several years of data) suggest that patient 14 has SVTs from 140-150 bpm and true VTs at rates above 160 bpm, parameter determination module 222 may set TDI at 160 bpm in updated IMD data. Accordingly, programmer 24 may program IMD 16 with TDI set to 160 bpm based on the first IMD data and longitudinal patient data 210, thus, updating TDI parameters of IMD 16 for patient 14.

Parameter determination module 222 may update NID parameters based on longitudinal patient data 210. For example, parameter determination module 222 may determine that NID parameters of the first IMD data are not optimal as compared to NID parameters suggested by longitudinal patient data 210 when NID parameters of the first IMD data differ from NID parameters suggested by longitudinal patient data 210 by a predetermined amount. Specifically, in one example, if the first IMD data includes NID parameters set to 12/16, but longitudinal patient data 210 suggests that several episodes (e.g., VT) of patient 14 are self terminating after diagnosis and before delivery of therapy, then parameter determination module 222 may extend NID, for example, to 18/24. In other words, if longitudinal patient data 210 indicates that episodes of patient 14 self terminate, then it may be beneficial to extend NID (e.g., to 18/24) to more readily allow an episode to self terminate without application of therapy. Accordingly, programmer 24 may program IMD 16 with an extended NID based on the first IMD data and longitudinal patient data 210, thus, updating NID parameters of IMD 16 for patient 14. Although NID parameters listed above include a ratio (e.g., 18/24) that may represent a percentage of consecutive cardiac intervals shorter than a threshold and be applicable to NID for VF, in other examples, a NID parameter, e.g., for VT, may include only a single number, e.g., 12 consecutive intervals shorter than a threshold.

Furthermore, parameter determination module 222 may update NID parameters of the first IMD data based on an effect the NID parameters may have on patient 14. For example, the first IMD data may not be optimal if parameter determination module 222 determines that longitudinal patient data 210 indicates that patient 14 experiences syncope at the current NID. Parameter determination module 222 may determine whether patient 14 experiences syncope at the NID of the first IMD data based on longitudinal patient data 210, such as activity, heart rate, intracardiac pressure, or respiration rate. If parameter determination module 222 determines that longitudinal patient data 210 indicates that patient 14 experiences syncope at the current NID, parameter determination module 222 may shorten NID. Accordingly, programmer 24 may program IMD 16 with a shortened NID based on the first IMD data and longitudinal patient data 210, thus, updating NID parameters of IMD 16 for patient 14.

Parameter determination module 222 may update values for an SVT and/or VT template, used for real-time wavelet analysis, for patient 14 based on longitudinal patient data 14. Parameter determination module 222 may compare the SVT and VT template of the first IMD data with an SVT and VT template, respectively, determined based on the longitudinal patient data 210. If parameter determination module 222 determines that the SVT and/or VT templates based on the longitudinal patient data 210 differ from the first IMD data, then parameter determination module 222 may recommend that the SVT and/or VT templates be updated. For example, parameter determination module 222 may determine an updated SVT and/or VT template based on EGMs in longitudinal data 210, then compare the updated templates with currently used templates to determine whether the current templates are optimal. If the current templates are not optimal, parameter determination module 222 may recommend that the templates be updated. The clinician may review the recommended SVT and/or VT templates on programmer 24 to determine whether to upload the templates to IMD 16.

Parameter determination module 222 may, based on analysis of longitudinal patient data 210, recommend modification to therapy parameter settings that control delivery of ATP. For example, parameter determination module 222 may determine that longitudinal patient data 210 suggests that ATP is more effective in treating patient VT episodes when various characteristics are present in the VT episodes. Characteristics may include: whether the episode is monomorphic or polymorphic, a cycle length of the episode, a variability in cycle length during the episode, characteristics of the onset of the episode (e.g., a rate of onset), stability during the episode, a specific morphology of the episode, an atrioventricular relationship during the episode, etc. Parameter determination module 222 may analyze longitudinal patient data 210 to determine for which characteristics ATP was effective, then parameter determination module 222 may recommend changes to therapy parameter settings based on the analysis. The clinician may review the recommended changes to the therapy parameter settings to determine whether to upload the therapy parameter settings to IMD 16.

Parameter determination module 222 may, based on analysis of longitudinal patient data 210, recommend modification to defibrillation pulse strengths. For example, parameter determination module 222 may determine that longitudinal patient data 210 suggests that pulses of a particular strength are more effective in treating patient VT/VF episodes when various characteristics are present in the VT/VF episodes. Characteristics corresponding to VT or VF may include: whether the episode is monomorphic or polymorphic, a cycle length of the episode, a variability in cycle length during the episode, characteristics of the onset of the episode (e.g., a rate of onset), stability during the episode, a specific morphology of the episode, an atrioventricular relationship during the episode, etc. Parameter determination module 222 may analyze longitudinal patient data 210 to determine which pulse strengths were effective in treating the patient based on episode characteristics, then parameter determination module 222 may recommend changes to settings of defibrillation pulse strengths based on the analysis. The clinician may review the recommended changes to the settings which control pulse strengths to determine whether to upload the settings to IMD 16.

In some examples, parameter determination module 222 may provide recommendations for updated parameter settings of IMD 16 relating to rate response based on analysis of at least one of activity threshold, activity acceleration, activity deceleration, and rate response slope. Such rate response parameters may benefit from optimizations that serve to provide the optimal pacing therapy to a patient during changes in activity level, e.g., sitting vs. walking. Modifications to rate response parameters may leverage data such as past patient activity, age, and rate response parameters of first IMD data to determine the rate response parameters of the updated IMD data. In some cases, comparison of rate response data to cross patient data 212 of similar patients may assist in determining if rate response settings in the updated IMD data may be inappropriate. e.g., too responsive, or not responsive enough.

Algorithm determination module 224 may modify various algorithms to be programmed into IMD 16. For example, algorithm determination module 224 may modify EGM comparison algorithms. In one example, if a current arrhythmia detection algorithm of IMD 16 compares a current EGM to a single waveform template, but algorithm determination module 224 determines, based on longitudinal patient data 210, that IMD 16 may perform more effectively if IMD 16 compares the current waveform to multiple templates, algorithm determination module 224 may modify the EGM comparison algorithm to compare the current waveform to multiple templates. Programmer 24 may then transfer the modified algorithm to IMD 16.

Algorithm determination module 224 may further modify EGM comparison algorithms based on longitudinal data 210 relating to cardiac cycle length. For example, based on longitudinal data 210, algorithm determination module 224 may determine whether patient 14 exhibits certain EGMs that correspond to certain cycle lengths. If patient 14 exhibits certain EGMs that correspond to certain cycle lengths, then algorithm determination module 224 may modify the EGM comparison algorithm of IMD 16 to compare the waveforms to certain templates (e.g., VT and/or SVT templates) based on a corresponding cycle length. Accordingly, algorithm determination module 224 may modify the EGM comparison algorithm to select different comparison templates based on longitudinal data 210. Subsequently, algorithm determination module 224 may transfer the modified EGM comparison algorithm to IMD 16.

In a similar manner, algorithm determination module 224 may modify other algorithms that algorithm determination module 224 determines, based on longitudinal patient data 210, to be more effective than algorithms that are currently implemented in IMD 16. For example, algorithm determination module 224 may modify atrial fibrillation detection algorithms. As a further example, algorithm determination module 224 may determine whether dynamic tachyarrhythmia discrimination algorithms are optimal for patient 14 based on longitudinal patient data 210. Dynamic tachyarrhythmia discrimination algorithms, which may include adding pacing into an arrhythmia to determine an origin of the arrhythmia, may be more effective in some patients, and the determination of whether or not to implement dynamic discrimination algorithms in IMD 16 may be made by algorithm determination module 224 based on longitudinal patient data 210.

In some examples, algorithm determination module 224 may determine whether to implement an algorithm in IMD 16 based on cross-patient data 212. For example, algorithm determination module 224 may determine that a certain algorithm may be more effective with patient 14 if the certain algorithm is effective in certain segments of the population having similar characteristics as patient 14, as indicated by cross-patient data 212. Algorithm determination module 224 may therefore suggest, based on cross-patient data 212, using the certain algorithm in IMD 16 when the certain algorithm is found to be more effective in segments of the population that have similar characteristics as patient 14. For example, algorithm determination module 224 may determine that a segment of the population has similar characteristics as patient 14 if the population has similar indications, comorbidities, similar frequencies of therapies delivered, similar types of arrhythmias present, or similar types of leads implanted.

In other examples, parameter determination module 222 and/or algorithm determination module 224 may, based on analysis of longitudinal data 210, provide updated recommendations for arrhythmia prevention algorithm settings or behavior based on analysis. For example, parameter determination module 222 may analyze flashback memory data within longitudinal data 210 to predict when VT might occur, and then make recommendations for arrhythmia prevention settings in the updated IMD data based on the analysis.

In some examples, parameter determination module 222 and/or algorithm determination module 224 may determine which detection vector (i.e., which electrodes and leads) provided the best historical performance with regard to detection of cardiac events, e.g., depolarizations, based on longitudinal patient data 210. Based on the determination, modules 222, 224 may generate updated IMD data that configures IMD 16 to use the best determined configuration.

In still other examples, parameter determination module 222 and/or algorithm determination module 224 may provide recommendations for changes to parameters or real-time algorithm behavior of IMD 16 based on analysis of data such as night and day heart rate. For example, day and night heart rate data collected by IMD 16 and stored in datastore 204 may be used to update the upper and lower heart rates used by IMD 16 to determine when the heart rate of patient 14 is out of an expected range at a particular time of day. The upper and lower heart rates may be used by IMD 16, for example, to alert a clinician that a patients nighttime heart rate is greater than an expected maximum.

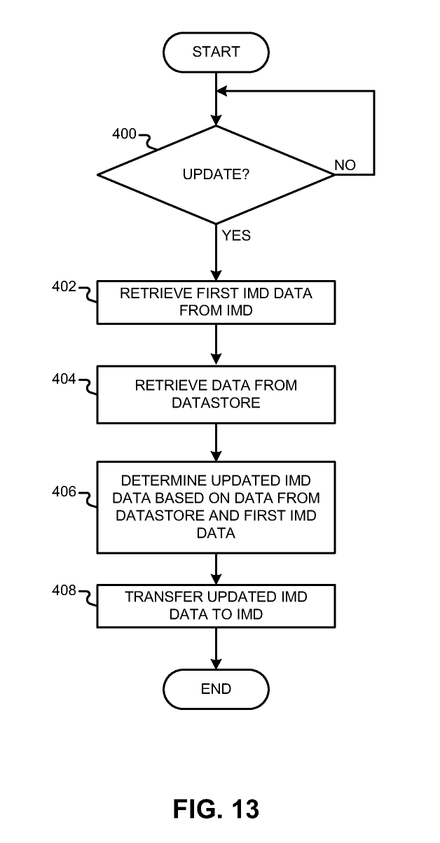

Referring now to FIG. 13, a method for updating IMD data during an update procedure is shown. Programmer 24 determines whether an update procedure is requested by the clinician (400). For example, the clinician may enter an update command in user interface 144 of programmer 24 to initiate an update procedure. Accordingly, programmer 24 may determine that an update is requested by the clinician upon receiving the update command. Programmer 24 retrieves first IMD data from IMD 16 in response to the update command (402). Remote device 200 retrieves data from datastore 204 (404). For example, data retrieved from datastore 204 may include longitudinal patient data 210 and/or cross-patient data 212. Remote device 200 determines updated IMD data based on the data retrieved from datastore 204 and the first IMD data received from programmer 24 (406). Specifically, remote device 200 may determine the updated IMD data based on at least one of the first IMD data, longitudinal patient data 210, or cross-patient data 212. Updated IMD data may include updated IMD parameters and/or updated IMD algorithms determined by remote device 200. Programmer 24 transfers the updated IMD data to IMD 16 (408).

Although the disclosure is described with respect to an implantable cardiac device, the techniques described may be applicable to other implantable devices, such as devices that provide spinal cord stimulation, deep brain stimulation, pelvic floor stimulation, gastric stimulation, occipital stimulation, functional electrical stimulation, and the like. Accordingly, the techniques described herein may be applicable to non-cardiac signals received from electrodes or sensors located on or within a patient.

The techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components. The term “processor” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the following claims.

1. A system comprising:
a data retrieval module that:
receives a command from a user, the command indicating a first implantable medical device (IMD) and a second IMD;
retrieves a first set of data from the first IMD in response to the command; and
retrieves a second set of data from a datastore, wherein the second set of data includes data retrieved from the first IMD and stored in the datastore prior to receiving the command; and
a determination module that:
determines a third set of data based on the first and second sets of data; and
transfers the third set of data to the second IMD.

2. The system of claim 1, wherein the second set of data includes data related to a patient having the first IMD.

3. The system of claim 2, wherein the second set of data includes data retrieved from the first IMD over a length of time during which the first IMD was implanted in the patient.

4. The system of claim 3, wherein the second set of data includes at least one of stored electrogram waveforms, stored marker channel data, stored lead impedance values associated with the first IMD, or stored heart rates of the patient.

5. The system of claim 3, wherein the second set of data includes data related to one or more detected arrhythmia episodes of the patient.

6. The system of claim 5, wherein the second set of data includes data related to at least one of ventricular fibrillation episodes of the patient, atrial tachycardia episodes of the patient, and atrial fibrillation episodes of the patient.

7. The system of claim 3, wherein the second set of data includes data related to one or more ventricular arrhythmia episodes of the patient, the data related to ventricular arrhythmia episodes indicating at least one of a cycle length of one of the ventricular arrhythmia episodes, a variability in cycle length during one of the ventricular arrhythmia episodes, characteristics of the onset of one of the ventricular arrhythmia episodes, whether one of the ventricular arrhythmia episodes was monomorphic or polymorphic, a stability associated with one of the ventricular arrhythmia episodes, a morphology of one of the ventricular arrhythmia episodes, a frequency of the ventricular arrhythmia episodes, and an atrioventricular relationship during one of the ventricular arrhythmia episodes.

8. The system of claim 2, wherein the second set of data includes programmable parameters of the first IMD.

9. The system of claim 8, wherein the programmable parameters include at least one of alert thresholds, detection intervals, or a number of intervals detected (NIDs).

10. The system of claim 9, wherein the third set of data includes at least one of alert thresholds, detection intervals, or NIDs.

11. The system of claim 2, wherein the second set of data includes data related to patients other than the patient having the first IMD.

12. The system of claim 11, wherein the data related to patients other than the patient having the first IMD includes data retrieved from IMDs of the patients other than the patient having the first IMD.

13. The system of claim 1, wherein the third set of data includes instructions to be executed by the second IMD.

14. The system of claim 13, wherein the instructions include at least one of an electrogram comparison algorithm, an atrial fibrillation detection algorithm, or a dynamic discrimination algorithm.

15. The system of claim 1, wherein the first IMD includes one of a cardiac pacemaker or a cardioverter-defibrillator, and wherein the second IMD includes one of a cardiac pacemaker or a cardioverter-defibrillator.

16. The system of claim 1, wherein the data retrieval module retrieves the second set of data from the datastore via at least one of the Internet or a wide area network.

17. A method comprising:
receiving a command from a user, the command indicating a first implantable medical device (IMD) and a second IMD;
retrieving a first set of data from the first IMD in response to the command;
retrieving a second set of data from a datastore, wherein the second set of data includes data retrieved from the first IMD and stored in the datastore prior to receiving the command;
determining a third set of data based on the first and second sets of data; and
transferring the third set of data to the second IMD.

18. The method of claim 17, wherein the second set of data includes data related to a patient having the first IMD.

19. The method of claim 18, wherein the second set of data includes data retrieved from the first IMD over a length of time during which the first IMD was implanted in the patient.

20. The method of claim 19, wherein the second set of data includes at least one of stored electrogram waveforms, stored marker channel data, stored lead impedance values associated with the first IMD, or stored heart rates of the patient.

21. The method of claim 19, wherein the second set of data includes data related to one or more detected arrhythmia episodes of the patient.

22. The method of claim 19, wherein the second set of data includes data related to one or more ventricular arrhythmia episodes of the patient, the data related to ventricular arrhythmia episodes indicating at least one of a cycle length of one of the ventricular arrhythmia episodes, a variability in cycle length during one of the ventricular arrhythmia episodes, characteristics of the onset of one of the ventricular arrhythmia episodes, whether one of the ventricular arrhythmia episodes was monomorphic or polymorphic, a stability associated with one of the ventricular arrhythmia episodes, a morphology of one of the ventricular arrhythmia episodes, a frequency of the ventricular arrhythmia episodes, and an atrioventricular relationship during one of the ventricular arrhythmia episodes.

23. The method of claim 18, wherein the second set of data includes programmable parameters of the first IMD.

24. The method of claim 23, wherein the programmable parameters include at least one of alert thresholds, detection intervals, or a number of intervals detected (NIDs).

25. The method of claim 24, wherein the third set of data includes at least one of alert thresholds, detection intervals, or NIDs.

26. The method of claim 18, wherein the second set of data includes data related to patients other than the patient having the first IMD.

27. The method of claim 26, wherein the data related to patients other than the patient having the first IMD includes data retrieved from IMDs of the patients other than the patient having the first IMD.

28. The method of claim 17, wherein the third set of data includes instructions to be executed by the second IMD.

29. The method of claim 28, wherein the instructions include at least one of an electrogram comparison algorithm, an atrial fibrillation detection algorithm, or a dynamic discrimination algorithm.

30. The method of claim 17, wherein the first IMD includes one of a cardiac pacemaker or a cardioverter-defibrillator, and wherein the second IMD includes one of a cardiac pacemaker or a cardioverter-defibrillator.

31. The method of claim 17, further comprising retrieving the second set of data from the datastore via at least one of the Internet or a wide area network.

32. A system comprising:
means for receiving a command from a user, the command indicating a first implantable medical device (IMD) and a second IMD;
means for retrieving a first set of data from the first IMD in response to the command;
means for retrieving a second set of data from a datastore, wherein the second set of data includes data retrieved from the first IMD and stored in the datastore prior to receiving the command;
means for determining a third set of data based on the first and second sets of data; and
means for transferring the third set of data to the second IMD.

33. The system of claim 32, further comprising means for retrieving the second set of data from the datastore via at least one of the Internet or a wide area network.

34. A method comprising:
receiving an update request;
retrieving a first set of data from an implantable medical device (IMD) implanted in a patient in response to the update request;
retrieving a second set of data from a datastore in response to the update request, wherein the second set of data includes data retrieved from the IMD and stored in the datastore prior to receiving the update request;
determining a third set of data based on the first and second sets of data; and
transferring the third set of data to the IMD.

35. The method of claim 34, wherein the second set of data includes data related to the patient having the IMD.

36. The method of claim 35, wherein the second set of data includes data retrieved from the IMD over a length of time during which the IMD was implanted in the patient.

37. The method of claim 36, wherein the second set of data includes at least one of stored electrogram waveforms, stored marker channel data, stored lead impedance values associated with the IMD, or stored heart rates of the patient.

38. The method of claim 36, wherein the second set of data includes data related to one or more detected arrhythmia episodes of the patient.

39. The method of claim 36, wherein the second set of data includes data related to one or more ventricular arrhythmia episodes of the patient, the data related to ventricular arrhythmia episodes indicating at least one of a cycle length of one of the ventricular arrhythmia episodes, a variability in cycle length during one of the ventricular arrhythmia episodes, characteristics of the onset of one of the ventricular arrhythmia episodes, whether one of the ventricular arrhythmia episodes was monomorphic or polymorphic, a stability associated with one of the ventricular arrhythmia episodes, a morphology of one of the ventricular arrhythmia episodes, a frequency of the ventricular arrhythmia episodes, and an atrioventricular relationship during one of the ventricular arrhythmia episodes.

40. The method of claim 35, wherein the second set of data includes programmable parameters of the IMD.

41. The method of claim 40, wherein the programmable parameters include at least one of alert thresholds, detection intervals, or a number of intervals detected (NIDs).

42. The method of claim 41, wherein the third set of data includes at least one of alert thresholds, detection intervals, or NIDs.

43. The method of claim 35, wherein the second set of data includes data related to patients other than the patient having the IMD.

44. The method of claim 42, wherein the data related to patients other than the patient having the IMD includes data retrieved from IMDs of the patients other than the patient having the IMD.

45. The method of claim 34, wherein the third set of data includes instructions to be executed by the IMD.

46. The method of claim 45, wherein the instructions include at least one of an electrogram comparison algorithm, an atrial fibrillation detection algorithm, or a dynamic discrimination algorithm.

47. The method of claim 34, wherein the IMD includes one of a cardiac pacemaker or a cardioverter-defibrillator.

48. The method of claim 34, further comprising retrieving the second set of data from the datastore via at least one of the Internet or a wide area network.
